(19) United States
(12) Patent Application Publication
(10) Pub. No.: US 2022/0287355 A1
(43) Pub. Date: Sep. 15, 2022

(54) ORAL PRODUCTS WITH SELF-EMULSIFYING SYSTEM

(71) Applicant: NICOVENTURES TRADING LIMITED, London (GB)

(72) Inventors: MICHAEL S. DANIEL, Winston-Salem, NC (US); THOMAS H. POOLE, Winston-Salem, NC (US)

(21) Appl. No.: 17/692,583

(22) Filed: Mar. 11, 2022

Related U.S. Application Data
(60) Provisional application No. 63/160,624, filed on Mar. 12, 2021.

(51) Int. Cl. A24B 13/00; A24B 15/10; A24B 15/40; A24B 15/30

(52) U.S. Cl. CPC A24B 13/00; A24B 15/10; A24B 15/406; A24B 15/308

(57) ABSTRACT

The disclosure provides solid products configured for oral use. The products contain a lipophilic active ingredient, a self-emulsifying delivery system (SEDS), a binder, and at least one sugar, at least one sugar alcohol, or a combination thereof. The SEDS is configured to provide droplets including the lipophilic active ingredient, the droplets have a D₉₀ value in a range from about 10 to about 1000 nm.

### CROSS-REFERENCE TO RELATED APPLICATIONS

This application claims priority to U.S. Provisional Application No. 63/160,624, filed on Mar. 12, 2022, and which is incorporated herein by reference in its entirety and for all purposes.

### FIELD OF THE DISCLOSURE

The present disclosure relates to an oral product. In particular, the present disclosure relates to compositions intended for human use. The compositions are configured for oral use and deliver an active ingredient during use.

### BACKGROUND

Tobacco may be enjoyed in a so-called “smokeless” form. Particularly popular smokeless tobacco products are employed by inserting some form of processed tobacco or tobacco-containing formulation into the mouth of the user. Conventional formats for such smokeless tobacco products include moist snuff, snus, and chewing tobacco, which are typically formed almost entirely of particulate, granular, or shredded tobacco, and which are either portioned by the user or presented to the user in individual portions, such as in single-use pouches or sachets. Other traditional forms of smokeless products include compressed or agglomerated forms, such as plugs, tablets, or pellets. Alternative product formats, such as tobacco-containing gums and mixtures of tobacco with other plant materials, are also known. See for example, the types of smokeless tobacco formulations, ingredients, and processing methodologies set forth in U.S. Pat. No. 1,376,586 to Schwartz; U.S. Pat. No. 4,513,756 to Pittman et al.; U.S. Pat. No. 4,528,993 to Sensabaugh, Jr. et al.; U.S. Pat. No. 4,624,269 to Story et al.; U.S. Pat. No. 4,991,599 to Tibbetts; U.S. Pat. No. 4,987,907 to Townsend; U.S. Pat. No. 5,092,352 to Sprinkle, III et al.; U.S. Pat. No. 5,387,416 to White et al.; U.S. Pat. No. 6,668,839 to Williams; U.S. Pat. No. 6,834,654 to Williams; U.S. Pat. No. 6,953,040 to Atchley et al.; U.S. Pat. No. 7,032,601 to Atchley et al.; and U.S. Pat. No. 7,694,686 to Atchley et al.; US Pat. Pub. Nos. 2004/0020503 to Williams; 2005/0115580 to Quinter et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0173317 to Robinson et al.; 2008/0209586 to Neilsen et al.; 2009/0065013 to Essen et al.; and 2010/0282267 to Atchley, as well as WO2004/095959 to Arnarp et al., each of which is incorporated herein by reference.

Smokeless tobacco product configurations that combine tobacco material with various binders and fillers have been proposed more recently, with example product formats including lozenges, pastilles, gels, extruded forms, and the like. See, for example, the types of products described in US Patent App. Pub. Nos. 2008/0196730 to Engstrom et al.; 2008/0305216 to Crawford et al.; 2009/0293889 to Kumar et al.; 2010/0291245 to Gao et al; 2011/0139164 to Mua et al.; 2012/0037175 to Cantrell et al.; 2012/0055494 to Hunt et al.; 2012/0138073 to Cantrell et al.; 2012/0138074 to Cantrell et al.;

2013/0074855 to Holton, Jr.; 2013/0074856 to Holton, Jr.; 2013/0152953 to Mua et al.; 2013/0274296 to Jackson et al.; 2015/0068545 to Moldoveanu et al.; 2015/0101627 to Marshall et al.; and 2015/0230515 to Lampe et al., each of which is incorporated herein by reference. Oral products in similar formats and which are free of tobacco have also been proposed.

It would be desirable to provide products configured for oral use which may deliver active ingredients to the consumer in an enjoyable form.

### BRIEF SUMMARY

In accordance with some embodiments described herein, there is provided an oral product comprising a lipophilic active ingredient and a self-emulsifying delivery system (SEDS). The SEDS is configured to provide droplets comprising the lipophilic active ingredient. Particularly, the SEDS is configured to form a nanoemulsion (i.e., micelles) in the mouth and gastrointestinal tract of the user upon use of the oral product, the droplets of which have a D₉₀ value in a range from about 10 to about 1000 nm. Without wishing to be bound by theory, it is believed that providing a lipophilic active ingredient in the form of such a nanoemulsion may result in one or more of the following: reduced lag time to systemic absorption, allow absorption via the lymphatic system, avoid first-pass metabolism, and increase the bioavailability of the lipophilic active ingredient.

Accordingly, in one aspect is provided a self-emulsifying delivery system (SEDS) comprising a lipophilic active ingredient, a lipid component, and an emulsifying agent, the SEDS configured to provide droplets comprising the lipophilic active ingredient.

In another aspect is provided an oral product comprising: a lipophilic active ingredient; a self-emulsifying delivery system (SEDS), configured to provide droplets comprising the lipophilic active ingredient; a binder; and at least one sugar, at least one sugar alcohol, or a combination thereof.

In some embodiments, the droplets have a D₉₀ value in a range from about 10 to about 1000 nm. In some embodiments, the droplets have a D₉₀ value in a range from about 10 to about 200 nm. In some embodiments, the droplets have a D₉₀ value in a range from about 40 to about 70 nm. In some embodiments, the droplets have a D₉₀ value from about 55 to about 65 nm.

In some embodiments, the SEDS comprises a lipid component and an emulsifying agent.

In some embodiments, the lipid component is a natural food-grade oil. In some embodiments, the natural food-grade oil comprises medium chain triglycerides. In some embodiments, the natural food-grade oil comprises long chain triglycerides. In some embodiments, the lipid component is sunflower oil.

In some embodiments, the emulsifying agent has an overall hydrophilic-lipophilic balance (HLB) value in a range from about 10 to about 20, about 11 to about 15, about 11 to about 14, or about 11 to about 13. In some embodiments, the emulsifying agent comprises a first emulsifier having an HLB value in a range from about 10 to about 20, and a second emulsifier having an HLB value in a range from about 1 to about 9. In some embodiments, the first emulsifier has an HLB value in a range from about 14 to 16. In some embodiments, the second emulsifier has an HLB value from about 2 to about 8, about 2 to 6, or from about 2 to about 4. In some embodiments, the emulsifying agent comprises glycerol monolineolate and a hydrogenated castor oil-polyethylene glycol polymer.

In some embodiments, the oral product further comprises an antioxidant. In some embodiments, the antioxidant has a logP value of about 3 or greater. In some embodiments, the antioxidant is a tocopherol, BHT, a fatty acid ester of vitamin C, or a combination thereof.

In some embodiments, the lipophilic active ingredient has a logP value of about 3 or greater. In some embodiments, the lipophilic active ingredient has a logP value in a range from about 4 to about 7. In some embodiments, the lipophilic active ingredient is selected from the group consisting of cannabinoids, cannabimimetics, terpenes, and combinations thereof. In some embodiments, the lipophilic active ingredient comprises cannabidiol (CBD). In some embodiments, the CBD is present in the oral product in an amount from about 15 mg to about 50 mg.

In some embodiments, the SEDS comprises up to about 15% of the oral product by weight. In some embodiments, the SEDS comprises from about 8 to about 14% of the oral product by weight.

In some embodiments, the oral product is in the form of a chew.

In some embodiments, the binder is selected from the group consisting of starches, gums, pectin, carrageenan, and combinations thereof. In some embodiments, the binder is a combination of pectin and carrageenan, the oral product optionally further comprising citric acid.

In some embodiments, the oral product comprises at least one sugar alcohol. In some embodiments, the at least one sugar alcohol comprises maltitol.

In some embodiments, the oral product further comprises one or more sweeteners, one or more flavorants, and water.

In another aspect is provided an oral product comprising: a lipophilic active ingredient selected from the group consisting of cannabinoids, cannabimimetics, terpenes, and combination thereof; a self-emulsifying delivery system (SEDS) in an amount from about 8 to about 14% of the oral product by weight based on the total weight of the oral product, the SEDS configured to provide droplets comprising the lipophilic active ingredient, and wherein the droplets have a D₉₀ value in a range from about 40 to about 70 nm, the SEDS comprising a food-grade oil, a first emulsifier having an HLB value in a range from about 14 to 16, and a second emulsifier having an HLB value in a range from about 2 to about 4; a binder selected from the group consisting of starches, gums, pectin, carrageenan, and combinations thereof; and sugar, at least one sugar alcohol, or a combination of at least one sugar and at least one sugar alcohol.

In some embodiments, the oral product is in the form of a chew.

The disclosure includes, without limitations, the following embodiments.

Embodiment 1: A self-emulsifying delivery system (SEDS) comprising a lipophilic active ingredient, a lipid component, and an emulsifying agent, the SEDS configured to provide droplets comprising the lipophilic active ingredient.

Embodiment 2: An oral product comprising: a lipophilic active ingredient; a self-emulsifying delivery system (SEDS), configured to provide droplets comprising the lipophilic active ingredient; a binder; and at least one sugar, at least one sugar alcohol, or a combination of at least one sugar and at least one sugar alcohol.

Embodiment 3: The oral product of embodiment 2, wherein the droplets have a D₉₀ value in a range from about 10 to about 1000 nm.

Embodiment 4: The oral product of embodiment 2 or 3, wherein the droplets have a D₉₀ value in a range from about 10 to about 200 nm.

Embodiment 5: The oral product of any one of embodiments 1-4, wherein the droplets have a D₉₀ value in a range from about 40 to about 70 nm.

Embodiment 6: The oral product of any one of embodiments 1-5, wherein the droplets have a D₉₀ value from about 55 to about 65 nm.

Embodiment 7: The oral product of any one of embodiments 1-6, wherein the SEDS comprises a lipid component and an emulsifying agent.

Embodiment 8: The oral product of any one of embodiments 1-7, wherein the lipid component is a food-grade oil.

Embodiment 9: The oral product of any one of embodiments 1-8, wherein the lipid component comprises long chain triglycerides.

Embodiment 10: The oral product of any one of embodiments 1-9, wherein the lipid component is sunflower oil.

Embodiment 11: The oral product of any one of embodiments 1-10, wherein the emulsifying agent has an overall hydrophilic-lipophilic balance (HLB) value in a range from about 10 to about 20, about 11 to about 15, about 11 to about 14, or about 11 to about 13.

Embodiment 12: The oral product of any one of embodiments 1-11, wherein the emulsifying agent comprises a first emulsifier having an HLB value in a range from about 10 to about 20, and a second emulsifier having an HLB value in a range from about 1 to about 9.

Embodiment 13: The oral product of any one of embodiments 1-12, wherein the first emulsifier has an HLB value in a range from about 14 to 16.

Embodiment 14: The oral product of any one of embodiments 1-13, wherein the second emulsifier has an HLB value from about 2 to about 8, about 2 to 6, or from about 2 to about 4.

Embodiment 15: The oral product of any one of embodiments 1-14, wherein the emulsifying agent comprises glycerol monolineolate and a hydrogenated castor oil-polyethylene glycol polymer.

Embodiment 16: The oral product of any one of embodiments 1-15, further comprising an antioxidant.

Embodiment 17: The oral product of any one of embodiments 1-16, wherein the antioxidant has a logP value of about 3 or greater.

Embodiment 18: The oral product of any one of embodiments 1-17, wherein the antioxidant is a tocopherol, BHT, a fatty acid ester of vitamin C, or a combination thereof.

Embodiment 19: The oral product of any one of embodiments 1-18, wherein the lipophilic active ingredient has a logP value of about 3 or greater.

Embodiment 20: The oral product of any one of embodiments 1-19, wherein the lipophilic active ingredient has a logP value in a range from about 4 to about 7.

Embodiment 21: The oral product of any one of embodiments 1-20, wherein the lipophilic active ingredient is selected from the group consisting of cannabinoids, cannabimimetics, terpenes, and combinations thereof.

Embodiment 22: The oral product of any one of embodiments 1-21, wherein the lipophilic active ingredient comprises cannabidiol (CBD).

Embodiment 23: The oral product of any one of embodiments 1-22, wherein the CBD is present in the oral product in an amount from about 15 mg to about 50 mg.

Embodiment 24: The oral product of any one of embodiments 1-23, wherein the SEDS comprises up to about 15% of the oral product by weight.

Embodiment 25: The oral product of any one of embodiments 1-24, wherein the SEDS comprises from about 8 to about 14% of the oral product by weight.

Embodiment 26: The oral product of any one of embodiments 1-25, in the form of a chew.

Embodiment 27: The oral product of any one of embodiments 1-26, wherein the binder is selected from the group consisting of starches, gums, pectin, carrageenan, and combinations thereof.

Embodiment 28: The oral product of any one of embodiments 1-27, wherein the binder is a combination of pectin and carrageenan, the oral product optionally further comprising citric acid.

Embodiment 29: The oral product of any one of embodiments 1-28, wherein the at least one sugar alcohol comprises maltitol.

Embodiment 30: The oral product of any one of embodiments 1-29, wherein the oral product further comprises one or more sweeteners, one or more flavorants, and water.

Embodiment 31: An oral product comprising: a lipophilic active ingredient selected from the group consisting of cannabinoids, cannabimimetics, terpenes, and combinations thereof; a self-emulsifying delivery system (SEDS) in an amount from about 8 to about 14% of the oral product by weight based on the total weight of the oral product, the SEDS configured to provide droplets comprising the lipophilic active ingredient, and wherein the droplets have a D₉₀ value in a range from about 40 to about 70 nm, the SEDS comprising a food-grade oil, a first emulsifier having an HLB value in a range from about 14 to 16, and a second emulsifier having an HLB value in a range from about 2 to about 4; a binder selected from the group consisting of starches, gums, pectin, carrageenan, and combinations thereof; an antioxidant having a logP value of about 3 or greater; at least one sugar, at least one sugar alcohol, or a combination of at least one sugar and at least one sugar alcohol; and one or more sweeteners, one or more flavorants, or a combination thereof.

Embodiment 32: The oral product of embodiment 31, in the form of a chew.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements.

As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water). Reference to “wet weight” refers to the weight of the composition including water. Unless otherwise indicated, reference to “weight percent” of a composition reflects the total wet weight of the composition (i.e., including water).

As described herein, there is provided an oral product comprising a lipophilic active ingredient and a self-emulsifying delivery system (SEDS). The SEDS is configured to provide droplets comprising the lipophilic active ingredient. The example individual components of the oral product, the overall oral product, and characteristics of the oral product are each described herein below.

### Self-Emulsifying Delivery System

Provided herein is a self-emulsifying delivery system (SEDS) as well as oral products comprising a SEDS. As used herein, the terms “self-emulsifying delivery system” or “SEDS” refer to a mixture of components which, when subject to moisture in the mouth of a user, in the gastrointestinal tract, or both, form a nanoemulsion. By “nanoemulsion” is meant a colloidal particulate system with particulates in the sub-micron size range, for example particulates having an average size of less than about 1,000 nm, such as from about 10 to about 1,000 nm. The particulates (referred to herein also as droplets) are generally spherical, and the surfaces of such droplets are hydrophilic and comprise an emulsifier, while the interiors are lipophilic and comprise a lipid component and lipophilic active ingredient, each as described herein.

The relative amount of SEDS present within the oral product may vary, and is typically selected so as to provide the desired sensory and performance characteristics to the overall oral product. In some embodiments, the oral product is in the form of a chew. According to the present disclosure, it has been discovered that the amount of SEDS present within the oral product in chew form is a determining factor in the physical properties of the chew. For example, smaller proportions of SEDS in the oral product lead to harder materials which are not chewable as defined herein, while larger proportions of SEDS lead to softer materials lacking the desired cohesiveness and resilience. In some embodiments, the oral product comprises up to about 15% by weight of the SEDS, such as from about 1% to about 15%, or from about 5 to about 10% by weight, based on the total weight of the oral product. In some embodiments, the oral product comprises from about 6 to about 10% SEDS by weight, based on the total weight of the oral product. In some embodiments, the oral product comprises about 8% SEDS by weight, based on the total weight of the oral product. The SEDS as disclosed herein generally comprises a lipid component, an emulsifying agent, and a lipophilic active ingredient.

### Lipid Component

The lipid component of the SEDS is generally an oil. Any suitable oil may be used, including petroleum-based (e.g., mineral oil) and natural or naturally derived oils (e.g., from plant materials or animal sources). Generally, the oil is a food-grade oil. Such oils include, but are not limited to, vegetable oils (e.g., acai oil, almond oil, amaranth oil, apricot oil, apple seed oil, argan oil, avocado oil, babassu oil, beech nut oil, ben oil, bitter gourd oil, black seed oil, black currant seed oil, borage seed oil, borneo tallow nut oil, bottle gourd oil, brazil nut oil, buffalo gourd oil, butternut squash seed oil, cape chestnut oil, canola oil, carob cashew oil, cocoa butter, cocklebur oil, coconut oil, corn oil, cothune oil, coriander seed oil, cottonseed oil, date seed oil, dika oil, egus seed oil, evening primrose oil, false flax oil, flaxseed oil, grape seed oil, grapefruit seed oil, hazelnut oil, hemp oil, kapok seed oil, kenaf seed oil, lallemantia oil, lemon oil, linseed oil, macadamia oil, mafura oil, marula oil, meadowfoam seed oil, mongongo nut oil, mustard oil, niger seed oil, nutmeg butter, okra seed oil, olive oil, orange oil, palm oil, palm stearin, papaya seed oil, peanut oil, pecan oil, perilla seed oil, persimmon seed oil, pequi oil, pili nut oil, pine nut oil, pistachio oil, pomegranate seed oil, poppyseed oil, pracaxi oil, prune kernel oil, pumpkin seed oil, quinoa oil, ramtil oil, rapeseed oil, rice bran oil, royle oil, sacha inchi oil, safflower oil, sapote oil, seje oil, sesame oil, shea butter, soybean oil, sunflower oil, taramira oil, tea seed oil, thistle oil, tigernut oil, tobacco seed oil, tomato seed oil, walnut oil, watermelon seed oil, wheat germ oil, and combinations thereof), animal oils (e.g., cattle fat, buffalo fat, sheep fat, goat fat, pig fat, lard, camel fat, tallow, liquid margarine, fish oil, fish liver oil, whale oil, seal oil, and combinations thereof), and mineral oils.

In some embodiments, the oil is a natural or naturally derived oil. In some embodiments, the oil comprises a long chain fatty acid or a medium chain fatty acid, such as the long chain fatty acid or medium chain fatty acid or one or more of a glycerol mono-, di-, or triester of either thereof (i.e., a monoacylglycerol, diacylglycerol, triacylglycerol, or a combination thereof, wherein the acyl group is a medium or long chain fatty acid).

As used herein, “medium chain fatty acid” refers to a carboxylic (CO₂H) acid having an aliphatic carbon chain of from about 6 to about 11 carbon atoms. The aliphatic carbon chain may be straight or branched. The aliphatic carbon chain may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the aliphatic carbon chain. Unsaturated alkyl groups may be mono- or polyunsaturated. Representative medium chain fatty acids include, but are not limited to, caproic acid, caprylic acid, decanoic acid, and undecanoic acid.

As used herein, “long chain fatty acid” refers to a carboxylic (CO2H) acid having an aliphatic carbon chain of from about 11 to about 21 carbon atoms. The aliphatic carbon chain may be straight or branched. The aliphatic carbon chain may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the aliphatic carbon chain. Unsaturated alkyl groups may be mono- or polyunsaturated. Representative long chain fatty acids include, but are not limited to undecylic acid, undecanoic acid, lauric acid, tridecanoic acid, myristic acid, pentadecanoic acid, palmitic acid, margaric acid, stearic acid, nonadecanoic acid, arachidic acid, heneicosanoic acid, α-linolenic acid, stearidonic acid, eicosapentaenoic acid, cervonic acid, linoleic acid, linolelaidic acid, γ-linolenic acid, dihomo-γ-linolenic acid, and arachidonic acid.

In some embodiments, the oil comprises a triacylglycerol, wherein the acyl group is a medium chain fatty acid as described herein. In some embodiments, the oil comprises a triacylglycerol, wherein the acyl group is a medium chain fatty acid as described herein. Such medium chain fatty acid triacylglycerols may also be referred to herein as “medium chain triglycerides” or “MCTs.” In some embodiments, the oil is enriched in MCT's, meaning the oil has a greater concentration of MCTs relative to other triglycerides, such as short or long chain triglycerides.

In some embodiments, the oil comprises a triacylglycerol, wherein the acyl group is a long chain fatty acid as described herein. Such long chain fatty acid triacylglycerols may also be referred to herein as “long chain triglycerides” or “LCTs.” In some embodiments, the oil is enriched in LCT's, meaning the oil has a greater concentration of LCTs relative to other triglycerides, such as short or medium chain triglycerides. In particular embodiments, the LCTs comprise a relatively large percentage of unsaturated long chain fatty acids (e.g., oleic and/or linoleic acids). Without wishing to be bound by any particular theory, it is believed that LCT's, and particularly LCTs containing a relatively large percentage of unsaturated fatty acids, may aid in promoting lymphatic transport of cannabinoid molecules present in the SEDS. One non-limiting example of an oil rich in LCTs, and specifically unsaturated fatty acid LCTs, is sunflower oil.

In some embodiments, the oil comprises castor oil, corn oil, coconut oil, cod liver oil, evening primrose oil, cottonseed oil, palm oil, rice bran oil, sesame oil, rapeseed oil, canola oil, cocoa butter, linseed oil, olive oil, peanut oil, soybean oil, safflower oil, flaxseed oil, sunflower oil, olive oil, or a combination thereof. In some embodiments, the oil is sunflower oil.

The amount of oil present within the SEDS can vary. In some embodiments, the SEDS comprises an oil in an amount of from about 1% to about 80% by weight, such as from about 5% to about 60% by weight, such as from about 5% to about 50% by weight, such as from about 5% to about 30% by weight, such as from about 10% to about 20% by weight, based on the total weight of the SEDS.

### Emulsifying Agent

The SEDS as disclosed herein comprises an emulsifying agent. By “emulsifying agent” is meant a substance which aids in the formation and stabilization of emulsions by promoting dispersion of hydrophobic and hydrophilic (e.g., oil and water) components. In general, emulsifying agents are amphiphilic molecules chosen from, for example, nonionic and ionic amphiphilic molecules. The expression “amphiphilic molecule” means any molecule of bipolar structure comprising at least one hydrophobic portion and at least one hydrophilic portion and having the property of reducing the surface tension of water and of reducing the interface tension between water and an oily phase. Emulsifying agents/amphiphilic molecules as provided herein are also referred to as, for example, surfactants and emulsifiers.

Suitable emulsifying agents include small molecule surfactants, phospholipids, proteins, polysaccharides, and mixtures thereof. Examples of suitable emulsifying agents include, but are not limited to, polyethylene glycol esters of fatty acids, propylene glycol esters of fatty acids, polysorbates, polyglycerol esters of fatty acids, polyglycerol polyricinoleate, sorbitan esters of fatty acid, sucrose esters of fatty acids, lecithins, enzyme treated lecithins, glycerin fatty acids esters, acetic acid esters of monoglycerides, lactic acid esters of monoglycerides, citric acid esters of monoglycerides, succinic acid esters of monoglycerides, diacetyl tartaric acid esters of monoglycerides, calcium stearoyl di lactate, chitin and chitosan derivatives, natural and modified starches, natural and modified hydrocolloids, natural and modified polysaccharides, natural and modified celluloses, natural and modified proteins, synthetic amphiphilic polymers, glycol distearate, sorbitan trioleate, sorbitan tristearate, sorbitan triisostearate, glyceryl isostearate, propylene glycol isostearate, glycol stearate, sorbitan sesquioleate, glyceryl stearate, lecithin, sorbitan oleate, sorbitan monostearate, sorbitan stearate, sorbitan isostearate, steareth-2, oleth-2, PEG-7 hydrogenated castor oil, laureth-2, sorbitan palmitate, laureth-3, glyceryl laurate, ceteth-2, PEG-30 dipolyhdroxystearate, glyceryl stearate SE, sorbitan stearate (and) sucrose cocoate, PEG-4 dilaurate, methyl glucose sesquistearate, PEG-8 dioleate, sorbitan laurate, PEG-40 sorbitan peroleate, laureth-4, PEG-7 glyceryl cocoate, PEG-20 almond glycerides, PEG-25 hydrogenated castor oil, stearamide MEA, glyceryl stearate (and) PEG-100 stearate, polysorbate 81, polysorbate 85, polysorbate 65, PEG-7 glyceryl cocoate, PEG-8 stearate, PEG-8 caprate, PEG-35 almond glycerides, PEG-6 laurate, laureth-7, steareth-10, isotrideceth-8, PEG-35 castor oil, isotrideceth-9, PEG-40 castor oil, ceteareth-12, laureth-9, PEG-40 hydrogenated castor oil, PEG-20 glyceryl isostearate, PEG-20 stearate, PEG-40 sorbitan perisostearate, PEG-7 olivate, cetearyl glucoside, PEG-8 oleate, polyglyceryl-3 methylglucose distearate, oleth-10, oleth-10/polyoxyl 10 oleyl ether NF, ceteth-10, PEG-8 laurate, cocamide MEA, polysorbate 60, polysorbate 80, isosteareth-20, PEG-60 almond glycerides, PEG-20 methyl glucose sesquistearate, ceteareth-20, oleth-20, steareth-20, steareth-21, ceteth-20, isoceth-20, polysorbate 20, polysorbate 40, ceteareth-25, ceteareth-30, PEG-30 stearate, laureth-23, PEG-75 lanolin, polysorbate 20, PEG-40 stearate, PEG-100 stearate, steareth-100, PEG-80 sorbitan laurate, polyoxyethylene stearate (e.g., polyoxyethylene (40) stearate), polyoxyethylene ether, and mixtures thereof.

In some embodiments, the emulsifying agent has an overall HLB value. As will be understood by one skilled in the art, HLB is the hydrophilic-lipophilic balance of an emulsifying agent and is a measure of the degree to which it is hydrophilic or lipophilic. The HLB value may be determined by calculating values for the different regions of the molecule, as described by Griffin in Griffin, William C. (1949), “Classification of Surface-Active Agents by ‘HLB’” (PDF), Journal of the Society of Cosmetic Chemists, 1 (5): 311-26 and Griffin, William C. (1954), “Calculation of HLB Values of Non-Ionic Surfactants” (PDF), Journal of the Society of Cosmetic Chemists, 5 (4): 249-56, and by Davies in Davies J T (1957), “A quantitative kinetic theory of emulsion type, I. Physical chemistry of the emulsifying agent” (PDF), Gas/Liquid and Liquid/Liquid Interface, Proceedings of the International Congress of Surface Activity, pp. 426-38. HLB value may be determined in accordance with the industry standard text book, namely “The HLB SYSTEM, a time-saving guide to emulsifier selection” ICI Americas Inc., Published 1976 and Revised, March, 1980. The HLB values of the emulsifiers described herein were determined in accordance with this standard method.

In some embodiments, the overall HLB value is in a range of from about 10 to about 20, such as from about 11 to about 15, about 11 to about 14, or about 11 to about 13. Such emulsifiers may be described as water-soluble or water-dispersible emulsifiers.

In some embodiments, the SEDS comprises at least two emulsifying agents which have different HLB values. In some embodiments, the SEDS comprises a first emulsifying agent with a high HLB value, and a second emulsifying agent with a low HLB value. In some embodiments, the SEDS comprises a first emulsifying agent having an HLB value from about 10 to about 20, about 12 to 18, about 13 to 17, or about 14 to about 16, and a second emulsifying agent having an HLB value from about 1 to about 9, such as from about 2 to about 8, about 2 to 6, or from about 2 to about 4. In some embodiments, the overall (i.e., combined) HLB value of the first and second emulsifying agents is from about 11 to about 15.

In embodiments where two emulsifying agents are present, the ratio of the first (high HLB) emulsifying agent to the second (low HLB) emulsifying agent may vary. For example, the ratio of the first to the second emulsifying agent may be from about 6:1 to about 1:1, such as about 6:1, about 5:1, about 4:1, about 3:1, about 2:1, about 1.6:1, about 1.5:1, about 1.4:1, about 1.3:1, about 1.2:1, about 1.1:1, or about 1:1.

In some embodiments, the first emulsifying agent is selected from any suitable emulsifying agent having an HLB value of from about 10 to about 20. Examples of suitable emulsifying agent having an HLB value in this range include, but are not limited to polyoxyethylene sorbitan fatty acid esters, hydrogenated castor oil ethoxylates, PEG mono- and di-esters of fatty acids, and fatty acid ethoxylates. In some embodiments, the first emulsifying agent is selected from the group consisting of laureth-4, PEG-7 glyceryl cocoate, PEG-20 almond glycerides, PEG-25 hydrogenated castor oil, stearamide MEA, glyceryl stearate (and) PEG-100 stearate, polysorbate 81, polysorbate 85, polysorbate 65, PEG-7 glyceryl cocoate, PEG-8 stearate, PEG-8 caprate, PEG-35 almond glycerides, PEG-6 laurate, laureth-7, steareth-10, isotrideceth-8, PEG-35 castor oil, isotrideceth-9, PEG-40 castor oil, ceteareth-12, laureth-9, PEG-40 hydrogenated castor oil, PEG-20 glyceryl isostearate, PEG-20 stearate, PEG-40 sorbitan perisostearate, PEG-7 olivate, cetearyl glucoside, PEG-8 oleate, polyglyceryl-3 methylglucose distearate, oleth-10, oleth-10/polyoxyl 10 oleyl ether NF, ceteth-10, PEG-8 laurate, cocamide MEA, polysorbate 60, polysorbate 80, isosteareth-20, PEG-60 almond glycerides, PEG-20 methyl glucose sesquistearate, ceteareth-20, oleth-20, steareth-20, steareth-21, ceteth-20, isoceth-20, polysorbate 20, polysorbate 40, ceteareth-25, ceteareth-30, PEG-30 stearate, laureth-23, PEG-75 lanolin, polysorbate 20, PEG-40 stearate, PEG-100 stearate, steareth-100, PEG-80 sorbitan laurate, polyoxyethylene stearate (e.g., polyoxyethylene (40) stearate), polyoxyethylene ether, and mixtures thereof.

In some embodiments, the second emulsifying agent is selected from any suitable emulsifying agent having an HLB value from about 1 to about 9. Examples of suitable emulsifying agents having an HLB value in this range include, but are not limited to, mono and diglycerydes of fatty acid including glyceryl stearate and glyceryl oleate; fatty acid esters of C12-C22 fatty alcohols including fatty acid esters of cetyl alcohol and fatty acid esters of stearoyl alcohol, mixtures of fatty acid esters of cetyl alcohol and fatty acid esters of stearoyl alcohol, mixtures of fatty acid esters of cetyl alcohol and fatty acid esters of stearoyl alcohol wherein the fatty acids are derived from olive oil (such as cetearyl olivate), fatty acid esters of sorbitol including sorbitan oleate, fatty acid esters of sorbitol wherein the fatty acids are derived from olive oil (such as sorbitan olivate or cetearyl olivate), and mixtures thereof. In some embodiments, the second emulsifying agent is selected from the group consisting of glycerol monolineolate, glycol distearate, sorbitan trioleate, sorbitan tristearate, sorbitan triisostearate, glyceryl isostearate, propylene glycol isostearate, glycol stearate, sorbitan sesquioleate, glyceryl stearate, lecithin (such as soy lecithin), sorbitan oleate, sorbitan monostearate, sorbitan stearate, sorbitan isostearate, steareth-2, oleth-2, PEG-7 hydrogenated castor oil, laureth-2, sorbitan palmitate, laureth-3, glyceryl laurate, ceteth-2, PEG-30 dipolyhdroxystearate, glyceryl stearate SE, PEG-4 dilaurate, methyl glucose sesquistearate, PEG-8 dioleate, sorbitan laurate, PEG-40 sorbitan peroleate, and mixtures thereof.

In some embodiments, the emulsifying agent is a combination of glycerol monolineolate and a hydrogenated castor oil-polyethylene glycol polymer. In some embodiments, the emulsifying agent is a combination of glycerol monolineolate and hydrogenated castor oil-PEG-40 polymer. An example of such a hydrogenated castor oil-PEG-40 polymer is Kolliphor® RH40, available from BASF, Inc.

The total amount of the emulsifying agent(s) present in the SEDS may vary. The total amount of the emulsifying agent(s) may be in a range of up to about 90% by weight, for example from about 50% to about 90%, such as about 50%, about 55%, about 60%, about 65%, about 70%, about 75%, about 80%, about 85%, or about 90% by weight, based on the entirety of the SEDS. In some embodiments, total amount of the emulsifying agent(s) present in the total oral product is from about 4% to about 11% by weight of the oral product, such as from about 5% to about 10% by weight of the oral product. In some embodiments, the oral product comprises from about 2.5 to about 6% by weight of Kolliphor® RH40 and from about 1.5 to about 2.5% by weight of glycerol monolineolate, based on the total weight of the oral product.

### Active Ingredient

The SEDS and oral product as disclosed herein includes one or more active ingredients. As used herein, an “active ingredient” refers to one or more substances belonging to any of the following categories: API (active pharmaceutical substances), food additives, natural medicaments, and naturally occurring substances that can have an effect on humans. Example active ingredients include any ingredient known to impact one or more biological functions within the body, such as ingredients that furnish pharmacological activity or other direct effect in the diagnosis, cure, mitigation, treatment, or prevention of disease, or which affect the structure or any function of the body of humans (e.g., provide a stimulating action on the central nervous system, have an energizing effect, an antipyretic or analgesic action, or an otherwise useful effect on the body). In some embodiments, the active ingredient may be of the type generally referred to as dietary supplements, nutraceuticals, “phytochemicals” or “functional foods”. These types of additives are sometimes defined in the art as encompassing substances typically available from naturally-occurring sources (e.g., botanical materials) that provide one or more advantageous biological effects (e.g., health promotion, disease prevention, or other medicinal properties), but are not classified or regulated as drugs.

Non-limiting examples of active ingredients include those falling in the categories of botanical ingredients (e.g., hemp, lavender, peppermint, eucalyptus, rooibos, fennel, cloves, chamomile, basil, rosemary, clove, citrus, ginger, cannabis, ginseng, maca, and tisanes), stimulants (e.g., caffeine or guarana), amino acids (e.g., taurine, theanine, phenylalanine, tyrosine, and tryptophan), vitamins (e.g., B6, B12, and C), antioxidants, nicotine components, pharmaceutical ingredients (e.g., nutraceutical and medicinal ingredients), cannabinoids (e.g., tetrahydrocannabinol (THC) or cannabidiol (CBD)) and/or melatonin. Each of these categories is further described herein below. The particular choice of active ingredients will vary depending upon the desired flavor, texture, and desired characteristics of the particular product.

The particular percentages of active ingredients present will vary depending upon the desired characteristics of the particular product. Typically, an active ingredient or combination thereof is present in a total concentration of at least about 0.001% by weight of the composition, such as in a range from about 0.001% to about 20%. In some embodiments, the active ingredient or combination of active ingredients is present in a concentration from about 0.1% w/w to about 10% by weight, such as, e.g., from about 0.5% w/w to about 10%, from about 1% to about 10%, from about 1% to about 5% by weight, based on the total weight of the composition. In some embodiments, the active ingredient or combination of active ingredients is present in a concentration of from about 0.001%, about 0.01%, about 0.1% , or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the composition. Further suitable ranges for specific active ingredients are provided herein below.

In some embodiments, the active ingredient is lipophilic. By lipophilic is meant that the active ingredient has the ability dissolve in fats, oils, lipids, and non-polar solvents. Lipophilicity is a key physicochemical parameter linking solubility, membrane permeability, and hence, absorption and distribution of substances within the body. Lipophilicity is conveniently measured in terms of logP, the partition coefficient of a molecule between a lipophilic phase and an aqueous phase, usually octanol and water, respectively. A positive logP value indicates that a molecule (e.g., an active ingredient) is more soluble in octanol than in water. In some embodiments, the lipophilic active ingredient has a logP value greater than about 3. In some embodiments, the lipophilic active ingredient has a logP value from about 4 to about 7, such as about 4, about 5, about 6, or about 7. Without wishing to be bound by theory, it is believed that certain lipophilic active ingredients may have greater absorption through the human gastrointestinal system when provided in the form of a nanoemulsion. Such nanoemulsified active ingredients may exhibit one or more of enhanced bioavailability, more rapid absorption, and reduced first-pass metabolism.

### Cannabinoids

In some embodiments, the lipophilic active ingredient comprises one or more cannabinoids. As used herein, the term “cannabinoid” refers to a class of diverse natural or synthetic chemical compounds that acts on cannabinoid receptors (i.e., CB1 and CB2) in cells that alter neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids). Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof. In some embodiments, the cannabinoid comprises at least tetrahydrocannabinol (THC). In some embodiments, the cannabinoid is tetrahydrocannabinol (THC). In some embodiments, the cannabinoid comprises at least cannabidiol (CBD). In some embodiments, the cannabinoid is cannabidiol (CBD). In some embodiments, the CBD is synthetic CBD. Notably, CBD has a logP value of about 6.5, making it insoluble in an aqueous environment (e.g., saliva). In some embodiments, the cannabinoid (e.g., CBD) is added to the oral product in the form of an isolate. An isolate is an extract from a plant, such as cannabis, where the active material of interest (in this case the cannabinoid, such as CBD) is present in a high degree of purity, for example greater than 95%, greater than 96%, greater than 97%, greater than 98%, or around 99% purity.

In some embodiments, the cannabinoid is an isolate of CBD in a high degree of purity, and the amount of any other cannabinoid in the oral product is no greater than about 1% by weight of the oral product, such as no greater than about 0.5% by weight of the oral product, such as no greater than about 0.1% by weight of the oral product, such as no greater than about 0.01% by weight of the oral product.

The choice of cannabinoid and the particular percentages thereof which may be present within the disclosed oral product will vary depending upon the desired flavor, texture, and other characteristics of the oral product.

In some embodiments, the cannabinoid (such as CBD) is present in the oral product in a concentration of at least about 0.001% by weight of the oral product, such as in a range from about 0.001% to about 2% by weight of the oral product. In some embodiments, the cannabinoid (such as CBD) is present in the oral product in a concentration of from about 0.1% to about 1.5% by weight, based on the total weight of the oral product. In some embodiments, the cannabinoid (such as CBD) is present in a concentration from about 0.4% to about 1.5% by weight, based on the total weight of the oral product.

Alternatively, or in addition to the cannabinoid, the lipophilic active agent may include a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids. Such compounds can be used in the same amounts and ratios noted herein for cannabinoids.

Terpenes

Lipophilic active ingredients suitable for use in the present disclosure can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

In some embodiments, the terpene is a terpene derivable from a phytocannabinoid producing plant, such as a plant from the stain of the cannabis sativa species, such as hemp. Suitable terpenes in this regard include so-called “C10” terpenes, which are those terpenes comprising 10 carbon atoms, and so-called “C15” terpenes, which are those terpenes comprising 15 carbon atoms. In some embodiments, the active ingredient comprises more than one terpene. For example, the active ingredient may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

### Other Active Ingredients

Other active ingredients which may be classified as lipophilic, or which may have lipophilic members within the family, include botanicals, amino acids, vitamins, antioxidants, stimulants, and pharmaceutical ingredients. In some embodiments, the oral product may further comprise one or more active ingredients which may be described as hydrophilic (i.e., more soluble in water than oils, fats, and lipids). Accordingly, the oral product may include a lipophilic active ingredient, and optionally may further include one or more hydrophilic active agents. In some embodiments, the lipophilic and/or hydrophilic active ingredient is selected from the group consisting of botanicals, amino acids, vitamins, antioxidants, stimulants, pharmaceutical ingredients, and combinations thereof. Each of these categories is described further herein below. One of skill in the art will recognize the lipophilic or hydrophilic nature of the disclosed active ingredients.

### Botanicals

In some embodiments, the active ingredient comprises or further comprises a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species).

When present, a botanical is typically at a concentration of from about 0.01% w/w to about 20% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, about 15%, about 16%, about 17%, about 18%, about 19%, or about 20% by weight, based on the total weight of the oral product.

The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include hemp, eucalyptus, rooibos, fennel, citrus, cloves, lavender, lemon balm, peppermint, chamomile, basil, rosemary, ginger, turmeric, green tea, white mulberry, cannabis, cocoa, ashwagandha, baobab, chlorophyll, cordyceps, damiana, ginseng, guarana, and maca.

In some embodiments, the active ingredient comprises or further comprises ashwagandha. Ashwagandha (Withania somnifera) is a plant in the Solanaceae (nightshade) family. As an herb, ashwagandha has found use in the Indian Ayurvedic system of medicine, where it is also known as “Indian Winter cherry” or “Indian Ginseng.”

In some embodiments, the active ingredient comprises or further comprises baobab. Baobab is the common name of a family of deciduous trees of the genus Adansonia. The fruit pulp and seeds of the Baobab are consumed, generally after drying, as a food or nutritional supplement.

In some embodiments, the active ingredient comprises or further comprises chlorophyll. Chlorophyll is any of several related green pigments found in the mesosomes of cyanobacteria, as well as in the chloroplasts of algae and plants. Chlorophyll has been used as a food additive (colorant) and a nutritional supplement. Chlorophyll may be provided either from native plant materials (e.g., botanicals) or in an extract or dried powder form.

In some embodiments, the active ingredient comprises or further comprises cordyceps. Cordyceps is a diverse genus of ascomycete (sac) fungi which are abundant in humid temperate and tropical forests. Members of the cordyceps family are used extensively in traditional Chinese medicine.

In some embodiments, the active ingredient comprises or further comprises damiana. Damiana is a small, woody shrub of the family Passifloraceae. It is native to southern Texas, Central America, Mexico, South America, and the Caribbean. Damiana produces small, aromatic flowers, followed by fruits that taste similar to figs. The extract from damiana has been found to suppress aromatase activity, including the isolated compounds pinocembrin and acacetin.

In some embodiments, the active ingredient comprises or further comprises guarana. Guarana is a climbing plant in the family Sapindaceae, native to the Amazon basin. The seeds from its fruit, which are about the size of a coffee bean, have a high concentration of caffeine and, consequently, stimulant activity.

In some embodiments, the active ingredient comprises or further comprises ginseng. Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the ginseng is American ginseng or Korean ginseng. In specific embodiments, the active ingredient comprises or further comprises Korean ginseng.

In some embodiments, the active ingredient comprises or further comprises lemon balm extract. Lemon balm (Melissa officinalis) is a mildly lemon-scented herb from the same family as mint (Lamiaceae). The herb is native to Europe, North Africa, and West Asia. The tea of lemon balm, as well as the essential oil and the extract, are used in traditional and alternative medicine.

In some embodiments, the active ingredient comprises maca. Maca is a plant that grows in central Peru in the high plateaus of the Andes Mountains. It is a relative of the radish, and has an odor similar to butterscotch. Maca has been used in traditional (e.g., Chinese) medicine.

### Stimulants

In some embodiments, the active ingredient comprises or further comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the oral product.

### Amino acids

In some embodiments, the active ingredient comprises or further comprises an amino acid. As used herein, the term “amino acid” refers to an organic compound that contains amine (—NH2) and carboxyl (—COOH) or sulfonic acid (SO3H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-₇-glutamyl ethyl amide), hydroxyproline, and beta-alanine.

When present, an amino acid or combination of amino acids (e.g., taurine, theanine, GABA, and combinations thereof) is typically at a concentration of from about 0.01% w/w to about 20% by weight, such as, e.g., from about 0.01, about 0.02, about 0.03, about 0.04, about 0.05, about 0.06, about 0.07, about 0.08, about 0.09, 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the oral product.

### Vitamins

In some embodiments, the active ingredient comprises or further comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones).

When present, a vitamin or combination of vitamins (e.g., vitamin B6, vitamin B12, vitamin E, vitamin C, or a combination thereof) is typically at a concentration of from about 0.0001% to about 6% by weight, such as, e.g., from about 0.0001, about 0.001, about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, about 1%, about 2%, about 3%, about 4%, about 5%, or about 6% by weight, based on the total weight of the oral product.

### Antioxidants

In some embodiments, the active ingredient comprises or further comprises one or more antioxidants. As used herein, the term “antioxidant” refers to a substance which prevents or suppresses oxidation by terminating free radical reactions, and may delay or prevent some types of cellular damage. Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, Yerba mate, Yerba santa, Bacopa monniera, Withania somnifera, Lion's mane, and Silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytoesterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12 (2005) 216-220, which is incorporated herein by reference.

Non-limiting examples of other suitable antioxidants include citric acid, Vitamin E or a derivative thereof, tocopherols, epicatechol, epigallocatechol, epigallocatechol gallate, propyl gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

In some embodiments, the SEDS or the overall oral product comprises an antioxidant. Without wishing to be bound by theory, it is believed that an antioxidant may help preserve certain lipophilic active ingredients, such, for example, cannabinoids and terpenes. In preferred embodiments, the antioxidant is a lipophilic antioxidant. By “lipophilic antioxidant” is meant that the antioxidant has a logP value of about 3 or greater. Without wishing to be bound by theory, it is believed that lipophilic antioxidants may better associate with the lipophilic active ingredient in the emulsion, the SEDS component, or both, to aid in protecting the lipophilic active ingredient from degradation. In some embodiments, the antioxidant comprises vitamin C or a derivative thereof, such as an ascorbate fatty acid ester; tocopherols; vitamin E or derivatives thereof; a flavonoid; a polyphenol; a carotenoid; a synthetic antioxidant such as BHT or BHA; or a combination thereof. In some embodiments, the antioxidant is selected from the group consisting of ascorbyl palmitate, butylated hydroxy anisole, butylated hydroxy toluene, propyl gallate, a-tocopherol, and y-tocopherol. In some embodiments, the SEDS or overall oral product comprises tocopherols as an antioxidant.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the oral product. In some embodiments, the overall oral product comprises from about 1 to about 1.5% by weight of tocopherols, based on the total weight of the oral product.

### Nicotine Component

In some embodiments, the oral product comprises a nicotine component. By “nicotine component” is meant any suitable form of nicotine (e.g., free base, salt, or ion pair) for providing oral absorption of at least a portion of the nicotine present. Nicotine is released from the composition and absorbed through the oral mucosa, thereby entering the blood stream, where it is circulated systemically.

Typically, the nicotine component is selected from the group consisting of nicotine free base, nicotine as an ion pair, and a nicotine salt. In some embodiments, at least a portion of the nicotine is in its free base form. In some embodiments, at least a portion of the nicotine is present as a nicotine salt, or at least a portion of the nicotine is present as an ion pair with at least a portion of the organic acid or the conjugate base thereof, as disclosed herein above.

Typically, the nicotine component (calculated as the free base) is present in a concentration of at least about 0.001% by weight of the composition, such as in a range from about 0.001% to about 10%. In some embodiments, the nicotine component is present in a concentration from about 0.1% w/w to about 10% by weight, such as, e.g., from about from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the oral product. In some embodiments, the nicotine component is present in a concentration from about 0.1% w/w to about 3% by weight, such as, e.g., from about from about 0.1% w/w to about 2.5%, from about 0.1% to about 2.0%, from about 0.1% to about 1.5%, or from about 0.1% to about 1% by weight, calculated as the free base and based on the total weight of the oral product.

### Pharmaceutical Ingredients

In some embodiments, the active ingredient comprises or further comprises a pharmaceutical ingredient. The pharmaceutical ingredient can be any known agent adapted for therapeutic, prophylactic, or diagnostic use. These can include, for example, synthetic organic compounds, proteins and peptides, polysaccharides and other sugars, lipids, inorganic compounds (e.g., magnesium, selenium, zinc, nitrate), neurotransmitters or precursors thereof (e.g., serotonin, 5-hydroxy-tryptophan, oxitriptan, acetylcholine, dopamine, melatonin), and nucleic acid sequences, having therapeutic, prophylactic, or diagnostic activity. Non-limiting examples of pharmaceutical ingredients include analgesics and antipyretics (e.g., acetylsalicylic acid, acetaminophen, 3-(4-isobutylphenyl)propanoic acid), phosphatidylserine, myoinositol, docosahexaenoic acid (DHA, Omega-3), arachidonic acid (AA, Omega-6), S-adenosylmethionine (SAM), beta-hydroxy-beta-methylbutyrate (HMB), citicoline (cytidine-5′-diphosphate-choline), and cotinine.

The amount of pharmaceutical ingredient may vary. For example, when present, a pharmaceutical ingredient is typically at a concentration of from about 0.001% w/w to about 10% by weight, such as, e.g., from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the oral product.

### CYP450 Inhibitors

In some embodiments, the SEDS or the overall oral product comprises an inhibitor of the cytochrome P450 (CYP450) enzyme system. The presence of certain CYP450 inhibitors may suppress metabolism of an active ingredient metabolized predominantly through a CYP450 enzyme mediated process. Known naturally occurring inhibitors of CYP450 enzymes include tobacco materials, grapefruit juice, and black pepper extracts. Without wishing to be bound by theory, it is believed that the presence of such inhibitors may have a synergistic effect in minimizing first pass metabolism of active ingredients including, but not limited to, CBD.

### Water

In some embodiments, the SEDS may comprise water. The water content of the SEDS may vary according to the desired properties of the oral product and the nanoemulsion produced upon use of the oral product. In some embodiments, the water content of the SEDS is up to about 10% by weight, based on the total weight of the SEDS. In some embodiments, the SEDS is substantially free of water, meaning no water has been added beyond trace amounts which may be present in components of the SEDS. For example, the SEDS may contain less than about 1%, less than about 0.1%, less than about 0.01%, less than about 0.001%, or even 0% water by weight.

### Properties of the Nanoemulsion

As described herein above, the self-emulsifying delivery system (SEDS) is configured to provide droplets comprising the lipophilic active ingredient. Upon exposure of the oral product comprising the SEDS to mouth conditions (e.g., moisture from saliva and optionally, mechanical chewing forces), the lipophilic active ingredient, lipid component, and emulsifying agent interact with water in saliva and optionally within the gastrointestinal tract to produce a nanoemulsion.

The nanoemulsion produced upon use of the oral product generally comprises nanoscale particles (or nanoscale droplets) having an average size (i.e., diameter) from about 10 nm to about 1,000 nm. “Average particle size” is synonymous with Dso, meaning half of the population of particles has a particle size above this point, and half below. D₉₀ particle size distribution indicates that 90% of the particles (by number) have a Feret diameter below a certain size. The size of nanoparticles may be determined by quasi-electric light scattering (QELS) as described in Bloomfield, Ann. Rev. Biophys. Bioeng., 10:421-450 (1981), incorporated herein by reference. It may also be measured by correlation spectroscopy that analyzes the fluctuation in scattering of light due to Brownian motion, or by transmission electron microscopy (TEM). Unless otherwise indicated, D₉₀ values reported herein are determined by light scattering using a nano-scale zetasizer (Malvern Zetasizer; Malvern Panalytical Inc., 117 Flanders Road, Westborough, Mass., USA).

In some embodiments, the D₉₀ particle size of the droplets as disclosed herein is from about 10 nm to about 200 nm, such as from about 20 nm to about 100 nm, or from about 40 nm to about 100 nm. In some embodiments, the D₉₀ particle size is about 100 nm, about 90 nm, about 80 nm, about 70 nm, about 60 nm, about 50 nm or about 40 nm. In some embodiments, the D₉₀ particle size is from about 40 nm to about 80 nm. In some embodiments, the D₉₀ particle size is from about 40 nm to about 70 nm. In some embodiments, the D₉₀ particle size is from about 50 nm to about 60 nm. Without wishing to be bound by theory, it is believed that droplets in this size range may be more readily absorbed through the intestinal lumen, may be available for lymphatic distribution, or both.

The nanoemulsion as described herein may be characterized by reference to a polydispersity index. Polydispersity indicates the uniformity of droplet size in a nanoemulsion. The higher the value of polydispersity, the lower will be the uniformity of droplet size. It may be defined as the ratio of standard deviation to mean droplet size, and may be measured by spectrophotometric methods. In some embodiments, the nanoemulsion has a polydispersity index of less than about 0.5.

The nanoemulsion as described herein may be characterized by reference to zeta potential. As appreciated by one skilled in the art, zeta potential is the measure of the electrical charge on particle surface in colloidal dispersions, for example, the charge on the surface of a droplet in the nanoemulsion. Zeta potential may be measured with a zeta analyser, for example a Malvern nano-series ZS Zetasizer. In some embodiments, the zeta potential of the nanoparticles is from about −50 mV to about +50 mV. In some embodiments, the zeta potential of the nanoparticles is from about −50 mV, about −40 mV, about −30 mV, about −20 mV, about −10 mV, or about 0 mV, to about 10 mV, about 20 mV, about 30 mV, about 40 mV, or about 50 mV.

### Oral Product

In some embodiments of the disclosure are provided an oral product comprising the SEDS as described herein, configured for insertion into the user's mouth (i.e., oral cavity). Such oral products comprising the SEDS further comprise a base material (i.e., a matrix) in order to provide desired characteristics to the oral product. Oral products as disclosed herein may take various forms, including gels, lozenges, tablets, melts, chews, pastilles, and gums. Generally, any suitable base material may be utilized to provide an oral product which is meltable, chewable, or otherwise dissolvable. In order to provide the desired physical and sensory properties of the oral product, the oral product generally comprises additional components such as sweeteners, binders, flavorants, and the like. Such additional components are further described herein below.

### Sweetener

In order to improve the sensory and/or physical properties of the oral product, one or more sweeteners may be added. The sweeteners can be any sweetener or combination of sweeteners, in natural or artificial form, or as a combination of natural and artificial sweeteners. Examples of natural sweeteners include fructose, sucrose, glucose, maltose, isomaltulose, mannose, galactose, lactose, stevia, honey, and the like. Examples of artificial sweeteners include sucralose, maltodextrin, saccharin, aspartame, acesulfame K, neotame and the like. In some embodiments, the sweetener is selected from the group consisting of fructose, sucrose, glucose, maltose, mannose, galactose, lactose, stevia, honey, sucralose, isomaltulose, maltodextrin, saccharin, aspartame, acesulfame K, neotame, erythritol, arabitol, ribitol, isomalt, maltitol, dulcitol, iditol, mannitol, xylitol, lactitol, sorbitol, and mixtures thereof. In some embodiments, the sweetener is selected from the group consisting of maltitol, xylitol, acesulfame K, aspartame, and mixtures thereof.

In some embodiments, the sweetener comprises one or more sugars. Suitable sugars include, but are not limited to, glucose, sucrose, fructose, maltose, isomaltulose, mannose, galactose, lactose.

In some embodiments, the sweetener comprises one or more sugar alcohols. Sugar alcohols are polyols derived from monosaccharides or disaccharides that have a partially or fully hydrogenated form.

Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). Sugar alcohols also provide certain characteristics to the oral product, for example, providing bulk, and in combination with other components (e.g., binder), provide physical attributes such as chewiness, firmness, softness, and the like. In some embodiments, the oral product is in the form of a chew, and comprises at least one sugar, at least one sugar alcohol, or a combination thereof. In some embodiments, the oral product in the form of a chew comprises at least one sugar alcohol. In some embodiments, the sugar alcohol is maltitol.

When present in the oral product, the sweetener or mixture of sweeteners may be present in an amount from about 50% to about 80% by weight of the oral product, such as about 50%, about 55%, about 60%, about 65%, about 70%, about 75%, or about 80% by weight, based on the total weight of the oral product.

### Binder

In some embodiments, the oral product comprises a binder. Binders generally function as thickening or gelling agents. Typical binders can be organic or inorganic, or a combination thereof.

Representative binders include cellulose derivatives (e.g., cellulose ethers), povidone, sodium alginate, starch-based binders, pectin, gums, carrageenan, pullulan, zein, and combinations thereof. In some embodiments, the oral product can be chewable, meaning the oral product has a mild resilience or “bounce” upon chewing, and possesses a desirable degree of malleability. An oral product in chewable form may be entirely dissolving, or may be in the form of a non-dissolving gum in which only certain components (e.g., active ingredients, flavor, sweetener) dissolve, leaving behind a non-dissolving matrix. Chewable embodiments generally include a binder, for example, a natural gum, starch, gelatin, pectin, or combination thereof.

Accordingly, in some embodiments, the oral product comprises a binder (or combination of binders) in an amount sufficient to provide the desired physical attributes and physical integrity to the oral product. The amount of binder utilized can vary, but is typically up to about 5% by weight, and certain embodiments are characterized by a binder content of at least about 0.1% by weight, such as from about 1% to about 5% by weight, or about 2% to about 4% by weight, based on the total weight of the oral product.

In certain embodiments, the binder includes a non-tobacco plant material or a derivative thereof. Non-limiting examples of derivatives of non-tobacco plant material include starches (e.g., from potato, wheat, rice, corn), natural cellulose, and modified cellulosic materials. “Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the composition based on the ability of the starch material to impart a specific organoleptic property to composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications, and are considered to be “genetically modified” starches. Other starches are obtained and subsequently modified by chemical, enzymatic, or physical means. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, acetylation, hydroxypropylation, and/or partial hydrolysis. Enzymatic treatment includes subjecting native starches to enzyme isolates or concentrates, microbial enzymes, and/or enzymes native to plant materials, e.g., amylase present in corn kernels to modify corn starch. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, and starch sodium octenyl succinate.

In certain embodiments, the binder includes a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, and combinations thereof.

In some embodiments, the binder comprises pectin. Pectins are natural polymers related to carbohydrates and which are acidic heteropolysaccharides (polysaccharides comprising multiple monosaccharide units). As opposed to carbohydrates, the pectin C-6 position contains a carboxylic acid (or corresponding methyl ester or carboxamide) group instead of a hydroxymethyl group. The principal subunit is known as galacturonic acid, which can be copolymerized with L-rhamnose. Other sugars are featured as side-chain substituents. Pectin acts as a thickening and gelling agent. Pectin isolated from sources such as apple pomace, citrus peels, sugarbeet waste from sugar manufacturing, sunflower heads discarded from seed harvesting, mango waste, and other commercially available pectins may be used. In combination with certain sugars, under acidic conditions (e.g., a pH of from about 2.5 to about 5), or in the presence of a gelation agent (calcium or other divalent alkaline earth elements), pectins may provide a gel or gum consistency to compositions as disclosed herein. In some embodiments, the binder comprises low methoxy pectin. Suitable low methoxy pectins include, for example, “GENU® pectin type LM-104 AS”, available from CP Kelco, Atlanta, Ga., USA. In some embodiments, the binder comprises low methoxy pectin in combination with a gelation agent. In some embodiments, the gelation agent comprises calcium ions, such as, but not limited to, calcium diphosphate. In some embodiments, the binder comprises a high methoxy pectin in combination with a suitable organic acid, such as citric acid. In some embodiments, the binder comprises a high methoxy pectin and the oral product further comprises citric acid, sodium citrate, or a combination thereof. In some embodiments, the binder comprises pectin, carrageenan, or a combination thereof. In some embodiments, the binder is a combination of pectin and carrageenan. A suitable example of a commercially available pectin-carrageenan combination is Ticagel GC 592, available from TIC Gums, White Marsh, Md., USA.

When present, a binder comprising pectin is typically present in an amount of up to about 4% by weight, for example, from about 1, about 1.5, about 2, about 2.5, about 3, about 3.5, or about 4% by weight, based on the total weight of the composition. For proper gelation of pectin-containing binders, typically an amount of an acid, such as citric acid, is present. For example, in some embodiments, the oral product comprises citric acid in an amount up to about 1%, such as from about 0.1 to about 1%, or about 0.5 to about 1% by weight, based on the total weight of the oral product. In some embodiments, the oral product further comprises sodium citrate as a buffer and/or additional pectin cross-linker. For example, in some embodiments, the oral product comprises sodium citrate in an amount up to about 1.5%, such as from about 0.5 to about 1.5%, or about 0.9 to about 1.3% by weight, based on the total weight of the oral product.

### Water

The moisture content (e.g., water content) of the oral product, prior to use by a consumer of the oral product, may vary according to the desired properties. Typically, the oral product, prior to insertion into the mouth of the user, is less than about 30% by weight of water, and generally is from about 15 to about 25% by weight of water, for example, from about 15 to about 25%, or about 15 to about 20% water by weight, based on the total weight of the oral product.

### Flavoring Agent

In some embodiments, the oral product comprises a flavorant. As used herein, the terms “flavor” and “flavorant” refer to materials which, where local regulations permit, may be used to create a desired taste, aroma or other somatosensorial sensation in a product for adult consumers. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy.

Flavoring agents may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. Flavoring agents may be imitation, synthetic or natural ingredients or blends thereof. Flavoring agents may be in any suitable form, for example, liquid such as an oil, solid such as a powder, or gas.

In some embodiments, the flavor may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucolyptol, WS-3.

In some embodiments, the flavor comprises menthol, spearmint and/or peppermint. In some embodiments, the flavorant is lipophilic. In some embodiments, the flavorant comprises a citrus oil. In some embodiments, the flavoring agent comprises a terpene.

The amount of flavorant utilized can vary, but is typically up to about 1% by weight, and certain embodiments are characterized by a flavoring agent content of at least about 0.1% by weight, such as about 0.1% to about 1% by weight, or about 0.5 to about 1% by weight, based on the total weight of the oral product.

### Taste Modifier

In some embodiments, the oral product comprises a taste modifying agent. In some embodiments, the taste modifier may mask the bitterness of the active ingredient, for example, a cannabinoid, in the oral product. The taste modifying agent may improve the organoleptic properties of an oral product as disclosed herein, and may serve to mask, alter, block, or improve e.g., the flavor of an oral product as disclosed herein. Non-limiting examples of such taste modifiers include analgesic or anesthetic herbs, spices, and flavors which produce a perceived cooling (e.g., menthol, eucalyptus, mint), warming (e.g., cinnamon), or painful (e.g., capsaicin) sensation. Certain taste modifiers fall into more than one overlapping category.

In some embodiments, the taste modifier modifies one or more of bitter, sweet, salty, or sour tastes. In some embodiments, the taste modifier targets pain receptors. In some embodiments, the cannabinoid has a bitter taste, and the oral product comprises a taste modifier which masks or blocks the perception of the bitter taste. In some embodiments, the taste modifier is a substance which targets pain receptors (e.g., vanilloid receptors) in the user's mouth to mask e.g., a bitter taste of another component (e.g., a cannabinoid). In some embodiments, the taste modifier is capsaicin.

In some embodiments, the taste modifier is the amino acid gamma-amino butyric acid (GABA), referenced herein above with respect to amino acids. Studies in mice suggest that GABA may serve function(s) in taste buds in addition to synaptic inhibition. See, e.g., Dvoryanchikov et al., J Neurosci. 2011 Apr. 13; 31 (15):5782-91. Without wishing to be bound by theory, GABA may suppress the perception of certain tastes, such as bitterness.

In some embodiments, the taste modifier is adenosine monophosphate (AMP). AMP is a naturally occurring nucleotide substance which can block bitter food flavors or enhance sweetness. It does not directly alter the bitter flavor, but may alter human perception of “bitter” by blocking the associated receptor.

In some embodiments, the taste modifier is lactisole. Lactisole is an antagonist of sweet taste receptors. Temporarily blocking sweetness receptors may accentuate e.g., savory notes.

When present, a representative amount of taste modifier is about 0.01% by weight or more, about 0.1% by weight or more, or about 1.0% by weight or more, but will typically make up less than about 10% by weight of the total weight of the oral product, (e.g., from about 0.01%, about 0.05%, about 0.1%, or about 0.5%, to about 1%, about 5%, or about 10% by weight of the total weight of the oral product).

### Organic Acid

In some embodiments, the oral product comprises as organic acid. When present, an organic acid may serve multiple purposes, including, but not limited to, gelation of pectin binders, as an ion pairing agent, as a flavorant, and as a preservative. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific composition ingredient as opposed to merely being inherently present as a component of another composition ingredient (e.g., the small amount of organic acid which may inherently be present in a composition ingredient, such as a tobacco material).

Suitable organic acids will typically have a range of lipophilicities (i.e., a polarity giving an appropriate balance of water and organic solubility). Typically, lipophilicities of suitable organic acids, as indicated by logP, will vary between about 1.4 and about 4.5 (more soluble in octanol than in water).

In some embodiments, the organic acid has a logP value of from about 1.5 to about 4.0, e.g., from about 1.5, about 2.0, about 2.5, or about 3.0, to about 3.5, about 4.0, about 4.5, or about 5.0. Particularly suitable organic acids have a logP value of from about 1.7 to about 4, such as from about 2.0, about 2.5, or about 3.0, to about 3.5, or about 4.0. In specific embodiments, the organic acid has a logP value of about 2.5 to about 3.5. In some embodiments, organic acids outside this range may also be utilized for various purposes and in various amounts, as described further herein below. For example, in some embodiments, the organic acid may have a logP value of greater than about 4.5, such as from about 4.5 to about 8.0. Particularly, the presence of certain solvents or solubilizing agents (e.g., inclusion in the composition of glycerin or propylene glycol) may extend the range of lipophilicity (i.e., values of logP higher than 4.5, such as from about 4.5 to about 8.0).

In some embodiments, the organic acid has a log P value of from about 1.4 to about 4.5, such as about 1.5, about 2, about 2.5, about 3, about 3.5, about 4 or about 4.5. In some embodiments, the organic acid has a log P value of from about 2.5 to about 3.5.

In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

As used herein, “alkyl” refers to any straight chain or branched chain hydrocarbon. The alkyl group may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the alkyl group. Unsaturated alkyl groups may be mono- or polyunsaturated. Representative straight chain alkyl groups include, but are not limited to, methyl, ethyl, n-propyl, n-butyl, n-pentyl, and n-hexyl. Branched chain alkyl groups include, but are not limited to, isopropyl, sec-butyl, isobutyl, tert-butyl, isopentyl, and 2-methylbutyl. Representative unsaturated alkyl groups include, but are not limited to, ethylene or vinyl, allyl, 1-butenyl, 2-butenyl, isobutylenyl, 1 -pentenyl, 2-pentenyl, 3-methyl-1-butenyl, 2-methyl-2-butenyl, 2,3-dimethyl-2-butenyl, and the like. An alkyl group can be unsubstituted or substituted.

“Cycloalkyl” as used herein refers to a carbocyclic group, which may be mono- or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a carbocyclic aromatic group. Examples of aryl groups include, but are not limited to, phenyl and naphthyl. An aryl group can be unsubstituted or substituted.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl group comprises up to 20 carbon atoms and from 1 to 3 heteroatoms selected from N, O, and S. A heteroaryl or heterocycloalkyl may be a monocycle having 3 to 7 ring members (for example, 2 to 6 carbon atoms and 1 to 3 heteroatoms selected from N, O, and S) or a bicycle having 7 to 10 ring members (for example, 4 to 9 carbon atoms and 1 to 3 heteroatoms selected from N, O, and S), for example: a bicyclo[4,5], [5,5], [5,6], or [6,6] system. Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Examples of heterocycloalkyls include by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl. Heteroaryl and heterocycloalkyl groups can be unsubstituted or substituted.

“Substituted” as used herein and as applied to any of the above alkyl, aryl, cycloalkyl, heteroaryl, heterocyclyl, means that one or more hydrogen atoms are each independently replaced with a substituent. Typical substituents include, but are not limited to, —Cl, Br, F, alkyl, —OH, —OCH₃, NH₂, —NHCH₃, —N(CH₃)₂, —CN, —NC(═O)CH₃, —C(═O)—, —C(═O)NH₂, and —C(═O)N(CH₃)₂. Wherever a group is described as “optionally substituted,” that group can be substituted with one or more of the above substituents, independently selected for each occasion. In some embodiments, the substituent may be one or more methyl groups or one or more hydroxyl groups.

In some embodiments, the organic acid is an alkyl carboxylic acid. Non-limiting examples of alkyl carboxylic acids include formic acid, acetic acid, propionic acid, butyric acid, valeric acid, caproic acid, heptanoic acid, octanoic acid, nonanoic acid, decanoic acid, undecanoic acid, dodecanoic acid, stearic acid, oleic acid, linoleic acid, linolenic acid, and the like.

In some embodiments, the organic acid is an alkyl sulfonic acid. Non-limiting examples of alkyl sulfonic acids include propanesulfonic acid, heptanesulfonic acid, and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like.

In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Further non-limiting examples of organic acids which may be useful in certain embodiments include 2,2-dichloroacetic acid, 2-hydroxyethanesulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-10-sulfonic acid (+), cinnamic acid, cyclamic acid, dodecylsulfuric acid, ethane-1,2-disulfonic acid, ethanesulfonic acid, furoic acid, galactaric acid, gentisic acid, glucoheptonic acid, gluconic acid, glucuronic acid, glutamic acid, glycerophosphoric acid, glycolic acid, hippuric acid, isobutyric acid, isovaleric acid, lactobionic acid, lauric acid, levulinic acid, malic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, and undecylenic acid.

Examples of suitable acids include, but are not limited to, the list of organic acids in Table 1.

TABLE 1
Non-limiting examples of suitable organic acids

| Acid Name | log(P) |
|---|---|
| benzoic acid | 1.9 |
| phenylacetic | 1.4 |
| p-toluic acid | 2.3 |
| ethyl benzoic acid | 2.9 |
| isopropyl benzoic acid | 3.5 |
| 4-phenylbutyric | 2.4 |
| 2-napthoxyacetic acid | 2.5 |
| napthylacetic acid | 2.7 |
| heptanoic acid | 2.5 |
| octanoic acid | 3.05 |
| nonanoic acid | 3.5 |
| decanoic acid | 4.09 |
| 9-deceneoic acid | 3.3 |
| 2-deceneoic acid | 3.8 |
| 10-undecenoic acid | 3.9 |
| dodecandioic acid | 3.2 |
| dodecanoic acid | 4.6 |
| myristic acid | 5.3 |
| palmitic acid | 6.4 |
| stearic acid | 7.6 |
| cyclohexanebutanoic acid | 3.4 |
| 1-heptanesulfonic acid | 2.0 |
| 1-octanesulfonic acid | 2.5 |
| 1-nonanesulfonic acid | 3.1 |
| monooctyl succinate | 2.8 |

In some embodiments, the organic acid is a mono ester of a di- or poly-acid, such as mono-octyl succinate, mono-octyl fumarate, or the like.

The selection of organic acid may further depend on additional properties in addition to or without consideration to the logP value. For example, an organic acid should be one recognized as safe for human consumption, and which has acceptable flavor, odor, volatility, stability, and the like. Determination of appropriate organic acids is within the purview of one of skill in the art.

In some embodiments, the organic acid is benzoic acid, a toluic acid, benzenesulfonic acid, toluenesulfonic acid, hexanoic acid, heptanoic acid, decanoic acid, or octanoic acid. In some embodiments, the organic acid is benzoic acid, octanoic acid, or decanoic acid. In some embodiments, the organic acid is octanoic acid.

In some embodiments, more than one organic acid may be present. For example, the composition may comprise two, or three, or four, or more organic acids. Accordingly, reference herein to “an organic acid” contemplates mixtures of two or more organic acids. The relative amounts of the multiple organic acids may vary. For example, a composition may comprise equal amounts of two, or three, or more organic acids, or may comprise different relative amounts. In this manner, it is possible to include certain organic acids (e.g., citric acid or myristic acid) which have a logP value outside the desired range, when combined with other organic acids to provide the desired average logP range for the combination. In some embodiments, it may be desirable to include organic acids in the composition which have logP values outside the desired range for purposes such as, but not limited to, providing desirable organoleptic properties, stability, as flavor components, and the like. Further, certain lipophilic organic acids have undesirable flavor and or aroma characteristics which would preclude their presence as the sole organic acid (e.g., in equimolar or greater quantities relative to nicotine). Without wishing to be bound by theory, it is believed that a combination of different organic acids may provide the desired ion pairing while the concentration of any single organic acid in the composition remains below the threshold which would be found objectionable from a sensory perspective.

For example, in some embodiments, the organic acid may comprise from about 1 to about 5 or more molar equivalents of benzoic acid relative to nicotine, combined with e.g., about 0.2 molar equivalents of octanoic acid or a salt thereof, and 0.2 molar equivalents of decanoic acid or a salt thereof.

In some embodiments, the organic acid is a combination of any two organic acids selected from the group consisting of benzoic acid, a toluic acid, benzenesulfonic acid, toluenesulfonic acid, hexanoic acid, heptanoic acid, decanoic acid, and octanoic acid. In some embodiments, the organic acid is a combination of benzoic acid, octanoic acid, and decanoic acid, or benzoic and octanoic acid. In some embodiments, the composition comprises citric acid in addition to one or more of benzoic acid, a toluic acid, benzenesulfonic acid, toluenesulfonic acid, hexanoic acid, heptanoic acid, decanoic acid, and octanoic acid.

In some embodiments, the composition comprises an alkali metal salt of an organic acid. For example, at least a portion of the organic acid may be present in the composition in the form of an alkali metal salt. Suitable alkali metal salts include lithium, sodium, and potassium. In some embodiments, the alkali metal is sodium or potassium. In some embodiments, the alkali metal is sodium. In some embodiments, the composition comprises an organic acid and a sodium salt of the organic acid.

In some embodiments, the composition comprises benzoic acid and sodium benzoate, octanoic acid and sodium octanoate, decanoic acid and sodium decanoate, or a combination thereof. In some embodiments, the ratio of the organic acid to the sodium salt of the organic acid is from about 0.1 to about 10, such as from about 0.1, about 0.25, about 0.3, about 0.5, about 0.75, or about 1, to about 2, about 5, or about 10. For example, in some embodiments, both an organic acid and the sodium salt thereof are added to the other components of the composition, wherein the organic acid is added in excess of the sodium salt, in equimolar quantities with the sodium salt, or as a fraction of the sodium salt. One of skill in the art will recognize that the relative amounts will be determined by the desired pH of the composition, as well as the desired ionic strength. For example, the organic acid may be added in a quantity to provide a desired pH level of the composition, while the alkali metal (e.g., sodium) salt is added in a quantity to provide the desired extent of ion pairing. As one of skill in the art will understand, the quantity of organic acid (i.e., the protonated form) present in the composition, relative to the alkali metal salt or conjugate base form present in the composition, will vary according to the pH of the composition and the pKa of the organic acid, as well as according to the actual relative quantities initially added to the composition.

The amount of organic acid or an alkali metal salt thereof present in the composition, relative to nicotine, may vary. Generally, as the concentration of the organic acid (or the conjugate base thereof) increases, the percent of nicotine that is ion paired with the organic acid increases. This typically increases the partitioning of the nicotine, in the form of an ion pair, into octanol versus water as measured by the logP (the logio of the partitioning coefficient). In some embodiments, the composition comprises from about 0.05, about 0.1, about 1, about 1.5, about 2, or about 5, to about 10, about 15, or about 20 molar equivalents of the organic acid, the alkali metal salt thereof, or the combination thereof, relative to the nicotine component, calculated as free base nicotine.

In some embodiments, the composition comprises from about 2 to about 10, or from about 2 to about 5 molar equivalents of the organic acid, the alkali metal salt thereof, or the combination thereof, to nicotine, on a free-base nicotine basis. In some embodiments, the organic acid, the alkali metal salt thereof, or the combination thereof, is present in a molar ratio with the nicotine from about 2, about 3, about 4, or about 5, to about 6, about 7, about 8, about 9, or about 10. In embodiments wherein more than one organic acid, alkali metal salt thereof, or both, are present, it is to be understood that such molar ratios reflect the totality of the organic acids present.

In certain embodiments the organic acid inclusion is sufficient to provide a composition pH of from about 4.0 to about 9.0, such as from about 4.5 to about 7.0, or from about 5.5 to about 7.0, from about 4.0 to about 5.5, or from about 7.0 to about 9.0. In some embodiments, the organic acid inclusion is sufficient to provide a composition pH of from about 4.5 to about 6.5, for example, from about 4.5, about 5.0, or about 5.5, to about 6.0, or about 6.5. In some embodiments, the organic acid is provided in a quantity sufficient to provide a pH of the composition of from about 5.5 to about 6.5, for example, from about 5.5, about 5.6, about 5.7, about 5.8, about 5.9, or about 6.0, to about 6.1, about 6.2, about 6.3, about 6.4, or about 6.5. In other embodiments, a mineral acid (e.g., hydrochloric acid, sulfuric acid, phosphoric acid, or the like) is added to adjust the pH of the composition to the desired value.

In some embodiments, the organic acid is added as the free acid, either neat (i.e., native solid or liquid form) or as a solution in, e.g., water, to the other composition components. In some embodiments, the alkali metal salt of the organic acid is added, either neat or as a solution in, e.g., water, to the other composition components. In some embodiments, the organic acid and the nicotine are combined to form a salt, either before addition to the composition, or the salt is formed within and is present in the composition as such. In other embodiments, the organic acid and nicotine are present as individual components in the composition, and form an ion pair upon contact with moisture (e.g., saliva in the mouth of the consumer).

### Salt

In some embodiments, the oral product comprises a salt (e.g., an alkali metal salt), typically employed in an amount sufficient to provide desired sensory attributes to the product. Non-limiting examples of suitable salts include sodium chloride, potassium chloride, ammonium chloride, flour salt, sodium acetate, sodium citrate, and the like.

When present, a representative amount of salt is at least about 0.5% by weight, such as at least about 1% by weight, such as at least about 1.5% by weight. In some embodiments, the oral product may comprise salt in an amount of from about 0.5% to about 10% by weight, such as from about 1% to about 7.5% by weight, such as from about 1.5% to about 5% by weight, based on the total weight of the oral product.

### Humectant

In certain embodiments, one or more humectants may be employed in the oral product of the present disclosure, and may be present in the SEDS, the overall oral product, or both. Examples of humectants include, but are not limited to, glycerin, 1,2-propanediol (propylene glycol), 1,3-propanediol, dipropylene glycol, sorbitol, xylitol, mannitol, and the like. In some embodiments, the oral product comprises glycerin. In some embodiments, the oral product comprises propylene glycol. In some embodiments, the oral product is substantially free of humectants.

Where included, the humectant is typically provided in an amount sufficient to provide desired moisture attributes to the oral product. Further, in some instances, the humectant may impart desirable flow characteristics to the oral product for depositing in a mold. When present, the humectant (such as glycerin and/or propylene glycol) may be present in an amount of from about 0.01% to about 10% by weight of the oral product.

### Buffering Agent

In certain embodiments, the oral product may comprise pH adjusters or buffering agents. Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Non-limiting examples of suitable buffers include alkali metals acetates, glycinates, phosphates, glycerophosphates, citrates, carbonates, hydrogen carbonates, borates, or mixtures thereof. In some embodiments, the buffering agent is selected from the group consisting of sodium citrate, sodium carbonate, sodium bicarbonate, sodium phosphate, ammonium phosphate, and mixtures thereof. In some embodiments, the buffering agent is sodium citrate.

Where present, the buffering agent is typically present in an amount less than about 5% based on the weight of the emulsion or the oral product comprising the emulsion; for example, from about 0.5% to about 5%, such as, e.g., from about 0.75% to about 4%, from about 0.75% to about 3%, or from about 1% to about 2% by weight, based on the total weight of the oral product.

### Oral Care Additives

In some embodiments, the composition comprises an oral care ingredient (or mixture of such ingredients). Oral care ingredients provide the ability to inhibit tooth decay or loss, inhibit gum disease, relieve mouth pain, whiten teeth, or otherwise inhibit tooth staining, elicit salivary stimulation, inhibit breath malodor, freshen breath, or the like. For example, effective amounts of ingredients such as thyme oil, eucalyptus oil and zinc (e.g., such as the ingredients of formulations commercially available as ZYTEX® from Discus Dental) can be incorporated into the composition. Other examples of ingredients that can be incorporated in desired effective amounts within the present composition can include those that are incorporated within the types of oral care compositions set forth in Takahashi et al., Oral Microbiology and Immunology, 19 (1), 61-64 (2004); U.S. Pat. No. 6,083,527 to Thistle; and US Pat. Appl. Pub. Nos. 2006/0210488 to Jakubowski and 2006/02228308 to Cummins et al. Other exemplary ingredients of tobacco containing-formulation include those contained in formulations marketed as MALTISORB® by Roquette and DENTIZYME® by NatraRx. When present, a representative amount of oral care additive is at least about 1%, often at least about 3%, and frequently at least about 5% of the total dry weight of the composition. The amount of oral care additive within the composition will not typically exceed about 30%, often will not exceed about 25%, and frequently will not exceed about 20%, of the total dry weight of the composition.

### Processing Aids

If necessary for downstream processing of the composition, such as granulation, mixing, or molding, a flow aid can also be added to the composition in order to enhance flowability of the composition. In some embodiments, the composition (e.g., melt and chew forms) may be surface treated with anti-stick agents, such as oils, silicones, and the like. Exemplary flow aids include microcrystalline cellulose, silica, polyethylene glycol, stearic acid, calcium stearate, magnesium stearate, zinc stearate, sodium stearyl fumarate, canauba wax, and combinations thereof. In some embodiments, the flow aid is sodium stearyl fumarate.

When present, a representative amount of flow aid may make up at least about 0.5 percent or at least about 1 percent, of the total dry weight of the composition. Preferably, the amount of flow aid within the composition will not exceed about 5 percent, and frequently will not exceed about 3 percent, of the total dry weight of the composition.

### Filler

The compositions as described herein may comprise one or more fillers. Fillers may fulfill multiple functions, such as enhancing certain organoleptic properties such as texture and mouthfeel, enhancing cohesiveness or compressibility of the product, and the like. In some cases, fillers can serve multiple functions, and therefore some components of the oral product can be considered both a filler and, for example, a sweetener or a binder.

The amount of filler can vary, but is typically greater than about 20%, and up to about 75% of the composition by weight, based on the total weight of the composition. A typical range of filler within the composition can be from about 20 to about 75% by total weight of the composition, for example, from about 20, about 25, or about 30, to about 35, about 40, about 45, or about 50% by weight (e.g., about 20 to about 50%, or about 25 to about 45% by weight). In certain embodiments, the amount of filler is at least about 20% by weight, such as at least about 25%, or at least about 30%, or at least about 35%, or at least about 40%, based on the total weight of the composition. In some embodiments, the amount of filler within the composition can be from about 38 to about 58% by total weight of the composition.

Generally, fillers are porous particulate materials and are cellulose-based. For example, suitable fillers are any non-tobacco plant material or derivative thereof, including cellulose materials derived from such sources. Examples of cellulosic non-tobacco plant material include cereal grains (e.g., maize, oat, barley, rye, buckwheat, and the like), sugar beet (e.g., FIBREX° brand filler available from International Fiber Corporation), bran fiber, and mixtures thereof. Non-limiting examples of derivatives of non-tobacco plant material include starches as described herein (e.g., from potato, wheat, rice, corn), natural cellulose, and modified cellulosic materials.

Additional examples of potential fillers include maltodextrin, dextrose, calcium carbonate, calcium phosphate, lactose, and sugar alcohols. Combinations of fillers can also be used. In some embodiments, the filler comprises or is a mixture of glucose and starch-derived polysaccharides. One such suitable mixture of glucose and starch-derived polysaccharides is EMDEX®, available from JRS PHARMA LP, USA, 2981 Route 22, Patterson, N.Y. 12563-2359.

In some embodiments, the filler comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). Isomalt is an equimolar mixture of two disaccharides, each composed of two sugars as follows: glucose and mannitol (α-D-glucopyranosido-1,6-mannitol); and glucose and sorbitol (α-D-glucopyranosido-1,6-sorbitol). In some embodiments, the one or more sugar alcohols comprise isomalt. In some embodiments, the one or more sugar alcohols is isomalt.

In some embodiments, the filler comprises a combination of isomalt and EMDEX®. In some embodiments, the one or more sugar alcohols is a combination of isomalt and EMDEX®.

In some embodiments, the one or more sugar alcohols is a combination of two or even three sugar alcohols. In some embodiments, the combination of sugar alcohols comprises or is isomalt and maltitol.

The total amount of sugar alcohols can vary, but is typically greater than about 30%, and up to about 95% of the composition by weight, based on the total weight of the composition. A typical range of sugar alcohols within the composition can be for example, from about 35, about 40, about 45, about 50, or about 55, to about 60, about 65, about 70, about 75, about 80, about 85, about 90, or about 95%, by weight. In certain embodiments, the amount of sugar alcohol is at least about 50% by weight, such as is at least about 55% by weight, or at least about 60%, or at least about 65%, or at least about 70%, or at least about 75%, or at least about 80%, or at least about 85%, based on the total weight of the composition.

In particular embodiments, the sugar alcohol is isomalt in an amount of from about 35 to about 55% by weight, based on the total weight of the composition, such as from about 35, about 40, or about 45, to about 50 or about 55% by weight.

In particular embodiments, the sugar alcohol is a combination of isomalt in an amount of from about 10 to about 25% by weight, such as about 10, about 15, about 20, or about 25% by weight; and maltitol in an amount of from about 50 to about 75% by weight, such as about 50, about 55, about 60, about 65%, about 70, about 75% by weight.

In particular embodiments, the filler is a combination of isomalt in an amount of from about 30 to about 50% by weight, based on the total weight of the composition, such as about 30, about 35, about 40, about 45, or about 50% by weight; and a glucose-polysaccharide blend (e.g., EMDEX) in an amount of from about 35 to about 55% by weight, based on the total weight of the composition, such as about 35, about 40, about 45, or about 50% by weight.

### Lipid

In some embodiments, the oral composition comprises a lipid (in addition to the lipid in the SEDS). Such compositions may, in some embodiments, be described as “meltable” or “melting” compositions, described further herein below. When present, the lipid of the composition is typically a fat, oil, or wax substance derived from animal or plant material (e.g., plant-derived fats), and typically comprises mostly triglycerides along with lesser amounts of free fatty acids and mono- or diglycerides. In certain embodiments, the lipid is a solid or semi-solid at room temperature (i.e., 25° C.) and capable of at least partially liquefying when subjected to the temperature of the oral cavity of the user (i.e., “melting”). Example plant-derived fats are comprised primarily of saturated or unsaturated fatty acid chains (most of which are bound within triglyceride structures) having a carbon length of about 10 to about 26 carbon atoms, or about 14 to about 20 carbon atoms, or about 14 to about 18 carbon atoms.

In some embodiments, the lipid comprises an oil and, in particular, a food grade oil including fractionated oils. In some embodiments, the lipid comprises a combination of oils. Such oils include, but are not limited to, vegetable oils (e.g., acai oil, almond oil, amaranth oil, apricot oil, apple seed oil, argan oil, avocado oil, babassu oil, beech nut oil, ben oil, bitter gourd oil, black seed oil, blackcurrant seed oil, borage seed oil, borneo tallow nut oil, bottle gourd oil, brazil nut oil, buffalo gourd oil, butternut squash seed oil, cape chestnut oil, canola oil, carob cashew oil, cocoa butter, cocklebur oil, coconut oil, corn oil, cothune oil, coriander seed oil, cottonseed oil, date seed oil, dika oil, egus seed oil, evening primrose oil, false flax oil, flaxseed oil, grape seed oil, grapefruit seed oil, hazelnut oil, hemp oil, kapok seed oil, kenaf seed oil, lallemantia oil, lemon oil, linseed oil, macadamia oil, mafura oil, manila oil, meadowfoam seed oil, mongongo nut oil, mustard oil, niger seed oil, nutmeg butter, okra seed oil, olive oil, orange oil, palm oil, papaya seed oil, peanut oil, pecan oil, perilla seed oil, persimmon seed oil, pequi oil, pili nut oil, pine nut oil, pistachio oil, pomegranate seed oil, poppyseed oil, pracaxi oil, prune kernel oil, pumpkin seed oil, quinoa oil, ramtil oil, rapeseed oil, rice bran oil, royle oil, sacha inchi oil, safflower oil, sapote oil, seje oil, sesame oil, shea butter, soybean oil, sunflower oil, taramira oil, tea seed oil, thistle oil, tigernut oil, tobacco seed oil, tomato seed oil, walnut oil, watermelon seed oil, wheat germ oil, and combinations thereof), animal oils (e.g., cattle fat, buffalo fat, sheep fat, goat fat, pig fat, lard, camel fat, tallow, liquid margarine, fish oil, fish liver oil, whale oil, seal oil, and combinations thereof), and mineral oils.

In certain embodiments, the plant-derived fats of the present disclosure include palm oil (including fractionated palm oil), palm kernel oil, soybean oil, cottonseed oil, and mixtures thereof. In one embodiment, the lipid is a blend of palm oil and palm kernel oil. The lipid can be, for example, hydrogenated, partially hydrogenated, or non-hydrogenated. Example embodiments of lipids can be purchased under the brand names CEBES®, CISAO®, or CONFAO®, available from AarhusKarlshamn USA Inc. or parent company, AAK AB.

The melting point of all or a portion of the lipid utilized in the composition is typically about 29° C. or above, such as about 29° C. to about 49° C., or about 36° C. to about 45° C., or about 38° C. to about 41° C. In some embodiments, use of lipids with a melting point of less than about 36° C. is not advantageous due to possible melting during product storage or handling. One test for determining the melting point of lipids is the Mettler dropping point method (ASTM D₃₉₅₄-15, Standard Test Method for Dropping Point of Waxes, ASTM International, West Conshohocken, Pa., 2015, www.astm.org.).

When present, the amount of lipid within the composition may vary. In certain embodiments, the amount of lipid is at least about 10 percent, at least about 20 percent, or at least about 30 percent, on a dry weight basis of the composition. In certain embodiments, the amount of lipid is less than about 70 percent, less than about 60 percent, or less than about 50 weight percent, on a dry weight basis. Example lipid weight ranges include about 10 to about 70 dry weight percent, such as about 35 to about 50 dry weight percent. In some embodiments, the amount of lipid is about 35, about 40, about 45, or about 50 percent by weight of the total composition. In some embodiments, the amount of lipid within the composition can be from about 35 to about 58% by total weight of the composition.

In some embodiments, the composition comprises a lipid. In one embodiment, the lipid is an oil selected from the group consisting of palm oil, palm kernel oil, soybean oil, sunflower oil, cottonseed oil, coconut oil, and combinations thereof, wherein the oil may be hydrogenated, partially hydrogenated, or non-hydrogenated. In one embodiment, the lipid is a fractionated non-hydrogenated cocoa butter substitute such as CEBES® 29-04 NH, available from AarhusKarlshanm USA Inc., 131 Marsh Street, Port Newark, N.J. 07114.

### Emulsifier

In certain embodiments, an emulsifier may be added to the base material (in addition to the SEDS). In some embodiments, the emulsifier is lecithin. For example, lecithin (e.g., soy lecithin or sunflower lecithin) may be added to the composition to provide smoother textural properties to the composition and to improve flowability and mixing of e.g., a lipid with the remaining components of the composition. Emulsifiers (e.g., lecithin) can be used in an amount of about 0.01 to about 5% by dry weight of the composition, such as from about 0.1 to about 2.5%, or from about 0.5 to about 1.5% based on the total weight of the composition.

### Other Additives

Other additives can be included in the oral product. For example, the oral product can be processed, blended, formulated, combined, and/or mixed with other materials or ingredients. The additives can be artificial, or can be obtained or derived from herbal or biological sources. Examples of further types of additives include thickening or gelling agents (e.g., fish gelatin), preservatives (e.g., potassium sorbate, sodium benzoate, calcium propionate, and the like), disintegration aids, zinc or magnesium salts selected to be relatively water soluble for oral products with greater water solubility (e.g., magnesium or zinc gluconate) or selected to be relatively water insoluble for oral products with reduced water solubility (e.g., magnesium or zinc oxide), or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., US Pat. App. Pub. No. 2010/0291245 to Gao et al., and US Pat. App. Pub. No. 2007/0062549 to Holton, Jr. et al., each of which is incorporated herein by reference. Typical inclusion ranges for such additional additives can vary depending on the nature and function of the additive and the intended effect on the final composition, with an example range of up to about 10% by weight, (e.g., from about 0.1% to about 5% by weight) based on total weight of the oral product

For example, where present, a preservative (such as potassium sorbate, sodium benzoate, calcium propionate, or the like) can be included in the oral product in an amount of from about 0.001% to about 5% by weight of the oral product, such as from about 0.01% to about 2.5% by weight of the oral product, such as from about 0.05% to about 1% by weight of the oral product.

A colorant may be employed in amounts sufficient to provide the desired physical attributes to the oral product. Examples of colorants include various dyes and pigments, such as caramel coloring and titanium dioxide. The amount of colorant utilized in the oral product can vary, but when present is typically up to about 3% by weight, such as from about 0.1%, about 0.5%, or about 1%, to about 3% by weight, based on the total weight of the oral product.

The aforementioned additives can be employed together (e.g., as additive formulations) or separately (e.g., individual additive components can be added at different stages involved in the preparation of the final product). Furthermore, the aforementioned types of additives may be encapsulated as provided in the final product or composition. Exemplary encapsulated additives are described, for example, in WO2010/132444 to Atchley, which is incorporated by reference herein.

### Configured for Oral Use

The oral products as described herein are configured for oral use. The term “configured for oral use” as used herein means that the product is provided in a form such that during use, saliva in the mouth of the user causes one or more of the components of the product (e.g., flavoring agents and/or active ingredients) to pass into the mouth of the user. In certain embodiments, the emulsion produced upon use of the product is adapted to deliver components to a user through mucous membranes in the user's mouth, the user's digestive system, or both, and, in some instances, said component is an active ingredient that can be absorbed through the mucous membranes in the mouth or absorbed through the digestive tract when the product is used.

The oral product as described herein is in a solid form. The oral product may take various forms, including chews, pastilles, gums, lozenges, and tablets. The textural properties of the oral product may vary. For example, a desired textural property may include one or more of adhesiveness, cohesiveness, density, dryness, fracturability, graininess, gumminess, hardness, heaviness, moisture absorption, moisture release, mouthcoating, roughness, slipperiness, smoothness, viscosity, and wetness. As used herein, the term “pastille” refers to a dissolvable oral product made by solidifying a liquid or gel composition, such as a composition that includes a gelling or binding agent, so that the final product is a hardened solid gel. As used herein, the terms “dissolve,” “dissolving,” and “dissolvable” refer to compositions having aqueous-soluble components that interact with moisture in the oral cavity and enter into solution, thereby causing gradual consumption of the product.

In particular embodiments, the mouthfeel of the oral product includes a slightly chewable and dissolvable quality. Such oral products are referred to herein as “chewable” or “chews.” By chewable is meant that the oral product has a mild resilience or “bounce” upon chewing, and possesses a desirable degree of malleability. Chewable oral products of the disclosure are characterized by sufficient cohesiveness to withstand light chewing action in the oral cavity without rapidly disintegrating. Chewable oral products of the disclosure typically do not exhibit a highly deformable chewing quality as found in conventional chewing gum. An oral product in chewable form may be entirely dissolving, or may be in the form of a non-dissolving gum in which only certain components (e.g., active ingredients, flavor, sweetener) dissolve, form a nanoemulsion, or both, leaving behind a non-dissolving matrix. According to certain embodiments, the oral products do not, to any substantial degree, leave any residue in the mouth of the user thereof, and do not impart a slick, waxy, or slimy sensation to the mouth of the user. Chewable embodiments generally include a binder, such as a natural gum, pectin, carrageenan, or combination thereof as described herein above. The oral product can be provided in any suitable predetermined shape or form, such as a general shape of a pill, pellet, tablet, coin, bead, ovoid, obloid, cube, or the like.

### Preparation of the Oral Product

The manner by which the various components of the oral product (e.g., lipid, emulsifier, active ingredient, binder, and the like) are combined may vary. As such, the overall oral product may be relatively uniform in nature (e.g., homogenous).

The various components of the oral product may be contacted, combined, or mixed together using any mixing technique or equipment known in the art. Any mixing method that brings the oral product ingredients into intimate contact can be used, such as a mixing apparatus featuring an impeller or other structure capable of agitation. Examples of mixing equipment include casing drums, conditioning cylinders or drums, liquid spray apparatus, conical-type blenders, ribbon blenders, mixers available as FKM130, FKM600, FKM1200, FKM2000 and FKM3000 from Littleford Day, Inc., Plough Share types of mixer cylinders, Hobart mixers, and the like. See also, for example, the types of methodologies set forth in U.S. Pat. No. 4,148,325 to Solomon et al.; U.S. Pat. No. 6,510,855 to Korte et al.; and U.S. Pat. No. 6,834,654 to Williams, each of which is incorporated herein by reference. In some embodiments, the components forming the oral product are prepared such that the mixture thereof may be used in a starch molding process for forming the oral product. In some embodiments, the components forming the oral product are prepared such that the mixture thereof may be used in a direct deposition molding process for forming the oral product. Manners and methods for formulating oral products will be apparent to those skilled in the art. See, for example, the types of methodologies set forth in U.S. Pat. No. 4,148,325 to Solomon et al.; U.S. Pat. No. Pat. No. 6,510,855 to Korte et al.; and U.S. Pat. No. 6,834,654 to Williams, U.S. Pat. No. 4,725,440 to Ridgway et al., and U.S. Pat. No. 6,077,524 to Bolder et al., each of which is incorporated herein by reference.

Generally, the SEDS component is prepared by combining a lipid (e.g., an oil), a lipophilic active ingredient, an emulsifying agent, and optionally a lipophilic antioxidant. The combination is mixed to form a homogenous mixture. In one non-limiting embodiment, a hydrophilic emulsifier is added to the lipophilic active ingredient, a lipid, a lipophilic emulsifier, and an antioxidant with stirring at an elevated temperature to form the SEDS component.

In some embodiments, the oral product is in chewable form. For the preparation of the oral product in chewable form, generally, the SEDS mixture is combined with water, binder, sweeteners including one or more sugars, sugar alcohols, or combinations thereof, flavor, and an acid at elevated temperature. The resulting composition is then deposited into molds for storage at ambient temperature, during which time the composition cools and solidifies, forming the oral product.

In some chew embodiments, a portion, for example, about 10%, of the total sweeteners (e.g., one or more sugars, sugar alcohols, or combinations thereof) may be dry mixed with a binder (e.g., a pectin-carrageenan mixture) and buffer (e.g., sodium citrate) to form a dry mix. The dry mix is then added to and mixed with heated water. The resulting suspension is brought to boiling for a period of time to provide a clear solution. The remaining sweetener is added to the resulting solution and the mixture heated and stirred for a period of time. The remaining sweetener is added and the temperature increased to about 100° C. with continued mixing. In some embodiments, the dissolved solids content (e.g., sugar alcohol) content is monitored by refractometry. In some embodiments, the mixing and heating is continued, with evaporation of water until a brix reading from 78 to 80 is obtained. The mixture is then cooled to slightly, for example, to about 90° C., and an acid (e.g., citric acid) and flavor is added. The solution is mixed until uniform, and the SEDS component added with stirring. The resulting composition is then deposited into molds. The molds may be starch molds or starchless molds. The chew composition may be held in the mold (starch or starchless) for a predetermined duration of time such as, for example, about 10 minutes to about 24 or even 48 hours, so as to allow the chew composition to cure and solidify.

In some embodiments, the composition is deposited in a starch mold. Starch trays with molded shapes are prepared and pre-heated at 60° C. for at least 1-2 hours. In some starch molded embodiments, the hot composition is deposited into the prepared starch molds for storage at ambient temperature. The resulting chews are removed from the starch mold, and any excess starch removed. In some starch molded embodiments, the hot composition deposited into the prepared starch molds and kept in an oven at 60° C. overnight, or until proper setting is achieved. The resulting chews are removed from the starch mold, and any excess starch removed. The starch can be any starch as disclosed herein above. In some embodiments, the starch is corn starch. In some embodiments, the chews are coated with CAPOL. In other embodiments, the composition is deposited in a starchless mold and left at room temperature, until proper setting is achieved. Such embodiments are advantageous from a time, labor, and cost standpoint relative to a starch molded embodiment, as there is no need for further drying, curing, or the like.

### EXAMPLES

Aspects of the present invention are more fully illustrated by the following examples, which are set forth to illustrate certain aspects of the present invention and are not to be construed as limiting thereof.

### General Procedures

To prepare SEDS mixtures, generally, a lipophilic active or combination of actives is dissolved in the lipid and/or lipophilic emulsifier, when present. When present, a hydrophilic emulsifier is then added and the mixture homogenized.

In some embodiments, weighed amounts of an oil and a low HLB emulsifier are mixed together. When present, a measured amount of an antioxidant is added to the mixing solution of oil and low HLB emulsifier. This solution is then heated to 50° C. The active ingredient is added to the heated solution while mixing, and stirring and heating continued until complete dissolution is observed. When present, a high HLB emulsifier is separately heated to 50° C. Once the active ingredient is fully dissolved and both solutions are at 50° C., the high HLB emulsifier is added into the oil/active/low HLB emulsifier/antioxidant solution. The resulting SEDS solution is either used directly (as in Example 1) or maintained at 50° C. until added to a chew base (as in Examples 3 and 4).

Particle size measurements for the emulsions were made on a Malvern Zetasizer Nano ZS. The sample material properties were set to a refractive index of ca. 1.395 and absorption of 0.010 (SEDS system properties measured on a standard refractometer) and default “water” setting as the dispersant. Samples were prepared by dispersing samples in water or otherwise specified solvent at about a 100:1 (SEDS only) or 10:1 (SEDS-containing chew embodiments) dispersant to sample ratio in 50 mL conical centrifuge tubes. Samples were agitated to achieve homogeneity and 2-5 mL aliquots were dispensed into 12 mm square polystyrene cuvettes. The samples were allowed to sit without disturbance in the instrument for 120 seconds. Measurements were taken in three sets of 12 averaged scans. The average of the three sets was recorded as the particle (micelle) size.

### Example 1. Screening of Self Emulsifying Delivery Systems

Approximately thirty SEDS formulations were prepared according to the general procedure using combinations of various lipids, emulsifying agents, and cosolvents, both in the presence and absence of lipophilic active ingredient (CBD, tocopherols). Lipids included no oil, olive oil, and sunflower oil.

Emulsifying agents included Kolliphor RH40, Poloxamer 188, glycerol monostearate, glycerol monooleate, glycerol monolinoleoate, and V-E TPGS (D-α-Tocopheryl polyethylene glycol 1000 succinate). Cosolvents included none, water, ethanol, or glycerol.

The various SEDS formulations were evaluated with respect to viscosity, and the following overall results were obtained. The combination of Poloxamer 188 and glycerol monostearate produced a solid SEDS. SEDS that formed a firm solid were considered less desirable in the context of incorporation into chews; specifically, when a SEDS including this combination was incorporated into a chew, the chew did not have a firm texture. Glycerol monostearate also produced a solid SEDS, even in the absence of Poloxamer 188. With respect to viscosity, the most favorable SEDS results were obtained with sunflower oil, glycerol monolinoleoate, and Kolliphor° RH40 in combination. Further, without wishing to be bound by any particular theory, it is believed that the sunflower oil (comprising unsaturated long-chain triglycerides) and glycerol monolinoleoate combination may be desirable with respect to the potential for enhanced lymphatic absorption and/or solubility of the lipophilic active ingredient for nanoemulsions of the present disclosure (e.g., including a lipophilic active ingredient).

### Example 2. SEDS Preparation and Evaluation

### Example 2A

A SEDS was prepared using the components and amounts provided in Table 2. When mixed with water, the SEDS produced micelles with a Dso average size of 28.9 nm and a z-average of 53.84 nm, as measured by a Malvern nano-series Zetasizer.

TABLE 2
SEDS Components and Amounts

| Component | % by weight of SEDS |
|---|---|
| Sunflower oil | 22 |
| glycerol monolinoleoate | 22 |
| Kolliphor ® RH40 | 46 |
| CBD | 9.4 |
| Tocopherols | 0.4 |

### Example 2B

A SEDS was prepared using the components and amounts provided in Table 3. When mixed with water, the SEDS produced micelles which were smaller than those of Example 2A.

TABLE 3
SEDS Components and Amounts

| Component | % by weight of SEDS |
|---|---|
| Sunflower oil | 21 |
| glycerol monolinoleoate | 21 |
| Kolliphor ® RH40 | 48.5 |
| CBD | 9.4 |
| Tocopherols | 0.4 |

### Example 3. Chew Preparation and Evaluation

Several SEDS were prepared using the components and amounts provided in Table 4.

TABLE 4
SEDS Components and Amounts

| Component | % by weight of SEDS |
|---|---|
| Olive oil | 21-27 |
| glycerol monooleoate | 21-27 |
| Kolliphor ® RH40 | 27-35 |
| CBD | 10-15 |
| Ethanol | 8-9 |

The prepared SEDS were subsequently incorporated in various amounts into chews containing water, maltitol syrup, pectin, carrageenan, citric acid, sweetener, and flavorant. Incorporation of 10% SEDS by weight, based on the total weight of the chew, created a chew that was too firm, while incorporation of 15% resulted in a chew that was too soft, based on an oral sensory evaluation.

### Example 4. Chew Preparation

A SEDS was prepared using CBD, Kolliphor® RH40, sunflower oil, glycerol monolinoleoate, and tocopherols in the amounts provided in Table 5. Kolliphor® RH40 was warmed in a water bath at 60° C. to ensure fluidity and homogeneity. The remaining SEDS ingredient were weighed and mixed together and heated to 50° C. The warmed Kolliphor® RH40 was added to the mixture of CBD, sunflower oil, glycerol monolinoleoate, and tocopherols with stirring at 200-300 rpm at 50° C. Approximately 10% of the total sweetener was dry mixed with the Ticagel® and sodium citrate. The dry mix was added to water heated to 90° C. and mixed. The suspension was brought to a boil for 3 minutes, providing a clear solution. The maltitol syrup, preheated to 85° C., was added to the Ticagel® solution and the mixture heated and stirred for 3 minutes. The remaining sweetener was added and the temperature increased to 100° C. with continued mixing. A sample was removed and placed in a refractometer. Once a reading of 78-80 Brix was obtained, the mixture was cooled to 90° C., and citric acid and flavor added. The solution was mixed until uniform, and the warm SEDS mixture added with stirring. The mixture was maintained at 90° C. while depositing in molds, which were then cooled to form the chews, each having a weight of approximately 3.6 grams.

TABLE 5
Chew Components and Amounts

| | % by Weight, based on the total |
|---|---|
| Ingredient | weight of chew |
| CBD | 0-1.5 |
| Kolliphor ® RH 40 | 2.5-6 |
| Glycerol Monolinoleate | 1.5-2.5 |
| Sunflower Oil | 1.5-2.5 |
| Tocopherols | 1-1.5 |
| Ticagel ® GC 592 | 2.5-4 |
| Water | 15-23 |
| Maltitol syrup | 40-60 |
| Sweetener | 12-18 |
| Flavor | 0.4-0.7 |
| Citric acid | 0.5-1.0 |
| Sodium citrate dihydrate | 0.9-1.3 |

The chews exhibited desirable texture, mouthfeel, and clarity, as evaluated by elasticity, bounce, firmness, absence of tooth stick, and no separation between the SEDS and the chewas a whole (i.e., the chew base comprising water, maltitol syrup, pectin, carrageenan, citric acid, sweetener, and flavorant). The chews were evaluated for self-emulsification. This was performed by dissolving the chew in a range of representative solvents (deionized water and simulated gastric fluid), The chew was suspended in the solvent at a weight ratio of 10:1 solvent/chew and stirred with a magnetic stir bar until the chew dissolved. A sample of the emulsion was withdrawn and analyzed on a Zetasizer. The droplets produced had a D₉₀ of approximately 60 nm.

1. An oral product comprising:
a lipophilic active ingredient;
a self-emulsifying delivery system (SEDS), configured to provide droplets comprising the lipophilic active ingredient;
a binder; and
at least one sugar, at least one sugar alcohol, or a combination of at least one sugar and at least one sugar alcohol.

2. The oral product of claim 1, wherein the droplets have a D₉₀ value in a range from about 10 to about 1000 nm.

3. The oral product of claim 1, wherein the droplets have a D₉₀ value in a range from about 10 to about 200 nm.

4. The oral product of claim 1, wherein the droplets have a D₉₀ value in a range from about 40 to about 70 nm.

5. The oral product of claim 1, wherein the droplets have a D₉₀ value from about 55 to about 65 nm.

6. The oral product of claim 1, wherein the SEDS comprises a lipid component and an emulsifying agent. 7 The oral product of claim 6, wherein the lipid component is a natural, food-grade oil.

8. The oral product of claim 7, wherein the natural food-grade oil comprises long chain triglycerides.

9. The oral product of claim 6, wherein the lipid component is sunflower oil.

10. The oral product of claim 1, wherein the emulsifying agent has an overall hydrophilic-lipophilic balance (HLB) value in a range from about 10 to about 20, about 11 to about 15, about 11 to about 14, or about 11 to about 13.

11. The oral product of claim 10, wherein the emulsifying agent comprises a first emulsifier having an HLB value in a range from about 10 to about 20, and a second emulsifier having an HLB value in a range from about 1 to about 9.

12. The oral product of claim 11, wherein the first emulsifier has an HLB value in a range from about 14 to 16.

13. The oral product of claim 11, wherein the second emulsifier has an HLB value from about 2 to about 8, about 2 to 6, or from about 2 to about 4.

14. The oral product of claim 1, wherein the emulsifying agent comprises glycerol monolineolate and a hydrogenated castor oil-polyethylene glycol polymer.

15. The oral product of claim 1, further comprising an antioxidant.

16. The oral product of claim 15, wherein the antioxidant has a logP value of about 3 or greater.

17. The oral product of claim 16, wherein the antioxidant is a tocopherol, BHT, a fatty acid ester of vitamin C, or a combination thereof.

18. The oral product of claim 1, wherein the lipophilic active ingredient has a logP value of about 3 or greater.

19. The oral product of claim 18, wherein the lipophilic active ingredient has a logP value in a range from about 4 to about 7.

20. The oral product of claim 1, wherein the lipophilic active ingredient is selected from the group consisting of cannabinoids, cannabimimetics, terpenes, and combinations thereof.

21. The oral product of claim 1, wherein the lipophilic active ingredient comprises cannabidiol (CBD).

22. The oral product of claim 21, wherein the CBD is present in the oral product in an amount from about 15 mg to about 50 mg.

23. The oral product of claim 1, wherein the SEDS comprises up to about 15% of the oral product by weight.

24. The oral product of claim 1, wherein the SEDS comprises from about 8 to about 14% of the oral product by weight.

25. The oral product of claim 1, in the form of a chew.

26. The oral product of claim 25, wherein the binder is selected from the group consisting of starches, gums, pectin, carrageenan, and combinations thereof.

27. The oral product of claim 25, wherein the binder is a combination of pectin and carrageenan, the oral product optionally further comprising citric acid.

28. The oral product of claim 1, wherein the at least one sugar alcohol comprises maltitol.

29. The oral product of claim 1, wherein the oral product further comprises one or more sweeteners, one or more flavorants, and water.

30. An oral product comprising:
a lipophilic active ingredient selected from the group consisting of cannabinoids, cannabimimetics, terpenes, and combinations thereof;
a self-emulsifying delivery system (SEDS) in an amount from about 8 to about 14% of the oral product by weight based on the total weight of the oral product, the SEDS configured to provide droplets comprising the lipophilic active ingredient, and wherein the droplets have a D₉₀ value in a range from about 40 to about 70 nm, the SEDS comprising a food-grade oil, a first emulsifier having an HLB value in a range from about 14 to 16, and a second emulsifier having an HLB value in a range from about 2 to about 4;
a binder selected from the group consisting of starches, gums, pectin, carrageenan, and combinations thereof;
an antioxidant having a logP value of about 3 or greater;
at least one sugar, at least one sugar alcohol, or a combination of at least one sugar and at least one sugar alcohol; and
one or more sweeteners, one or more flavorants, or a combination thereof.

31. The oral product of claim 30, in the form of a chew.
